(19) United States
(12) Patent Application Publication
(10) Pub. No.: US 2011/0165102 A1
(43) Pub. Date: Jul. 7, 2011

(54) COSMETIC COMPOSITION WITH IMPROVED APPLICATION TIME

(75) Inventors: Stephane Arditty, Ballainvilliers (FR); Nathalie Jager Lezer, Verrieres-Le-Buisson (FR)

(73) Assignee: L'OREAL, Paris (FR)

(21) Appl. No.: 12/999,197

(22) PCT Filed: Jul. 21, 2009
(86) PCT No.: PCT/IB09/53156

§ 371 Date: Feb. 2, 2011

Related U.S. Application Data
(60) Provisional application No. 61/086,534, filed on Aug. 6, 2008.

(30) Foreign Application Priority Data
Jul. 21, 2008 (FR) 08 54942

(51) Int. Cl. A61K 8/92; A61Q 1/10; A45D 40/26

(52) U.S. Cl. 424/70.7; 424/70.1; 424/70.11; 424/70.13; 424/70.122; 424/70.6; 132/218

(57) ABSTRACT

The invention relates to an anhydrous cosmetic composition for making up and/or caring for the eyelashes and/or the eyebrows, comprising at least one volatile hydrocarbon solvent of C₉-C₁₅ volatile linear alkane(s) type having an evaporation rate of less than or equal to 0.13 mg/cm²/min, and comprises less than 25% by weight of non-volatile oil relative to the total weight of the composition and less than 35% by weight of surfactant relative to the total weight of the composition.

The present invention relates to cosmetic compositions for making up and/or caring for keratin fibres, in particular the eyelashes, the eyebrows and the hair, and more particularly for making up the eyelashes.

Makeup compositions for the eyes, also called “mascara” for the eyelashes and “eye-liner” for the eyelids, are generally constituted of a wax or a mixture of waxes dispersed, using at least one surfactant, in an aqueous phase containing, moreover, water-soluble polymers and pigments.

Two types of mascara can be differentiated according to their formulation: washable mascaras which can be cleaned off with water or soapy water and which are generally provided in the form of an emulsion of waxes in water, such as creams or gels, and waterproof mascaras, requiring the use of an oily formulation in order to be removed and which are generally formulated in the form of a dispersion of wax in organic solvents.

Moreover, the application specificities desired for makeup compositions, for instance their fluidity, their covering capacity and/or their curling capacity, are adjusted through the qualitative and quantitative choice of the waxes and of the polymers. Thus, it is possible to prepare various compositions which, when applied in particular to the eyelashes, induce varied effects of the lengthening, curling and/or thickening (charging effect) and/or volumizing type.

Therefore, “waterproof” mascaras have good staying power, but may produce a lesser volumizing effect than washable mascaras.

The charging nature of mascaras, their volumizing nature, and the staying power of the material deposited on the eyelashes are often sought for the purpose of emphasizing the look and of making it more visible and intense throughout the day.

With the currently available makeup compositions, these effects are generally obtained by superimposition of several layers of makeup compositions on the keratin fibres, and more particularly the eyelashes.

These effects also require the use of compositions having a sufficiently high capacity for adhesion or attachment to the eyelashes.

For example, Application US 2003/031637 describes waterproof mascaras which are transfer-resistant and comprise a volatile hydrocarbon-based solvent.

In addition, these compositions should have a sliding capacity and be pleasant to apply.

However, the need to go over the eyelashes several times in a row with a composition that has strong adhesion can result in the eyelashes sticking together, thus forming packets, and resulting in an unattractive effect.

What is more, these compositions should be easy to apply and should have a considerable play-time on application, or application time. In particular, these compositions should have a long drying time in order to facilitate multiple passes.

Currently, many mascaras, in particular volumizing mascaras, have the drawback of having a weak play-time on application, or application time, over the course of the passes of the brush.

Moreover, the waterproof mascaras which are formulated to be water-resistant, may, because of this specific formulation, be difficult to remove and cause irritations.

There thus exists a need to be able to formulate cosmetic compositions, especially for making up keratin fibres, in particular waterproof mascaras, having a sustained play-time on application, or application time.

There also exists a need to be able to provide cosmetic compositions, especially for making up keratin fibres, in particular waterproof mascaras, having an increased drying or evaporation time, enabling numerous passes.

There also exists a need to provide cosmetic compositions, especially for making up keratin fibres, in particular waterproof mascaras, having a high adhesion or attachment capacity while at the same time maintaining a satisfactory, or even improved, manageability and comfort on application.

There also exists a need to have cosmetic compositions, especially for making up keratin fibres, in particular waterproof mascaras, having an improved staying power over time.

There also exists a need to have cosmetic compositions, especially for making up keratin fibres, in particular waterproof mascaras, of which the fluidity and viscosity can be adjusted so as to confer thereon easy application while at the same time having a satisfactory, or improved, staying power over time.

There also exists a need to have cosmetic compositions, especially making up keratin fibres, in particular waterproof mascaras, capable of generating an improved volumizing effect.

Finally, there also exists a need to have waterproof cosmetic compositions, in particular waterproof mascaras, which remain easy to remove and do not cause irritations.

An object of the present invention is to meet these needs.

Thus, according to one of these first objects, the invention relates to an anhydrous cosmetic composition for making up and/or caring for the eyelashes and/or the eyebrows, comprising at least one volatile hydrocarbon solvent of C₉-C₁₅ volatile linear alkane(s) type having an evaporation rate of less than or equal to 0.13 mg/cm²/min, said composition comprising less than 25% by weight of non-volatile oil relative to the total weight of the composition and less than 35% by weight of surfactant relative to the total weight of the composition.

For the purpose of the invention, the term “anhydrous” is intended to mean a composition comprising a water content of less than 10%, in particular less than 5%, more particularly less than 2%, and more particularly less than 1% by weight, relative to the total weight of the composition, or which is even devoid of water.

Unexpectedly, the inventors have observed that the use, in an anhydrous cosmetic composition, of at least one volatile hydrocarbon solvent of C₉-C₁₅ volatile linear alkane(s) type having a specific volatility, and in particular a lower volatility than the hydrocarbon solvents normally used in cosmetic compositions, such as isododecane, makes it possible to confer on these compositions a sustained application time and an improved ease of application.

In particular, the inventors have observed, as illustrated by the examples, that the use of at least one volatile hydrocarbon solvent of C₉-C₁₅ volatile linear alkane(s) type, such as, for example, n-undecane or n-tridecane or a mixture thereof, makes it possible to confer on compositions comprising less than 25% by weight of non-volatile oil and less than 35% by weight of surfactant, an improved play-time, or application time, while at the same time preserving cosmetic properties and comfort on application which are satisfactory, or even also improved.

The compositions of the invention advantageously have an improved volumizing effect and an improved staying power over time.

Advantageously, mascaras using hydrocarbon solvents of C₉-C₁₅ volatile linear alkane(s) type according to the invention may be provided with an improved capacity for individualizing the eyelashes.

In addition, the compositions of the invention have the advantage of conferring a more flexible aspect on keratin fibres.

According to another advantage, the compositions of the invention may advantageously exhibit the architecture of a cream, facilitating their application and conferring on them an improved comfort on application.

According to a variant embodiment, a volatile hydrocarbon solvent of C₉-C₁₅ volatile linear alkane(s) type that is suitable for the invention can be selected from volatile linear alkanes.

For the purpose of the present invention, the term “linear alkane” is understood to mean a non-branched linear alkane in contrast with branched alkanes.

According to another of its aspects, the present invention relates to the cosmetic use of at least one volatile hydrocarbon solvent of C₉-C₁₅ volatile linear alkane(s) type having an evaporation rate of less than or equal to 0.13 mg/cm²/min, for conferring on said composition a sustained application time.

A subject of the present invention is also an assembly for making up and/or caring for the eyelashes, comprising at least one composition according to the invention and at least one applicator of the composition, said applicator comprising means for smoothing out and/or separating the eyelashes.

A subject of the present invention is also a cosmetic process for making up and/or caring for keratin fibres, in particular the eyelashes, or a support, comprising at least the application to said fibres, in particular to said eyelashes, or said support, of at least one layer of a composition according to the invention.

A cosmetic process of the invention may in particular be implemented by means of an assembly in accordance with the invention.

A composition of the invention may be a dispersion.

The present invention relates to cosmetic compositions that may be in the form of a composition for caring for keratin fibres, especially the eyelashes and the eyebrows, and in particular the eyelashes. It may then be in an uncoloured form, optionally containing cosmetic active agents or keratin-care active agents. It may then be used as a care base for keratin fibres, in particular the eyebrows or the eyelashes.

A composition of the invention may also be in the form of a coloured product for making up keratin fibres, in particular for making up the eyelashes or the eyebrows, such as a mascara.

A composition according to the invention is in particular, waterproof.

According to the invention, the term “keratin fibres” is intended to denote the entire hair system, and in particular the hair, the eyelashes or the eyebrows, and more particularly the eyelashes.

For the purpose of the present invention, the term “keratin fibres” also extends to synthetic false eyelashes and hairpieces.

Play-Time or Application Time

For the purpose of the invention, the terms “play-time” and “application time”, used in an interchangeable manner, denote a composition with the property of being able to be easily and comfortably applied for a more or less long period of time. In particular, these terms denote the time necessary to obtain a deposit of makeup which is dry to the touch on the surface to be made up.

The play-time, or application time, can be evaluated by means of the following protocol.

The composition is applied to the support, in particular to the keratin fibres (in vivo), preferably the eyelashes, or to a test sample of false eyelashes (in vitro), and the time necessary to obtain a deposit that is dry to the touch is measured. The dry deposit corresponds to a deposit which no longer transfers to the fingers.

Volatile Hydrocarbon Solvents of Volatile Linear Alkane(s) Type

A composition of the invention comprises at least one volatile hydrocarbon solvent of volatile linear alkane(s) type, the volatile hydrocarbon solvent of volatile linear alkane(s) type being defined as follows.

For the purpose of the present invention, the expression “volatile hydrocarbon solvent of volatile linear alkane(s) type” is understood to mean a solvent comprising a single compound of volatile linear alkane type or a mixture of compounds of volatile linear alkane type.

A volatile hydrocarbon solvent of volatile linear alkane(s) type is essentially composed of volatile linear alkane(s) being described as follows.

The term “essentially” is understood to mean that the volatile hydrocarbon solvent of volatile linear alkane(s) type contains at least 80% by weight, preferably at least 90% by weight and even at least 95% or even 98% by weight of volatile linear alkane(s) relative to the total weight of hydrocarbon in said solvent.

A volatile hydrocarbon solvent of volatile linear alkane(s) type belongs to the liquid fatty phase of the composition of the invention, in particular to the hydrocarbon oil phase, and more particularly to the hydrocarbon-based oil phase.

A volatile hydrocarbon solvent of volatile linear alkane(s) type suitable for the invention has an evaporation rate of less than or equal to 0.13 mg/cm²/min.

According to one embodiment, a volatile hydrocarbon solvent of volatile linear alkane(s) type suitable for the invention can have an evaporation rate included within the range of from 0.05 to 0.13 mg/cm²/min, in particular from 0.08 to 0.12 mg/cm²/min, and more particularly from 0.1 to 0.12 mg/cm²/min.

The volatility of a volatile hydrocarbon solvent of volatile linear alkane(s) type in accordance with the invention can in particular be evaluated by means of the protocol described in WO 06/013413, and more particularly by means of the protocol described hereinafter.

15 g of volatile hydrocarbon solvent of volatile linear alkane(s) type are introduced into a crystallizing dish (diameter: 7 cm) placed on a balance located in a chamber of approximately 0.3 m³ with a regulated temperature (25° C.) and hygrometry (50% relative humidity).

The liquid is allowed to evaporate freely, without stirring, ventilation being provided by means of a fan (Papst-Motoren, reference 8550 N, at a spin speed of 2700 rpm) arranged vertically above the crystallizing dish containing the volatile hydrocarbon solvent of volatile linear alkane(s) type, the blades being directed towards the crystallizing dish and 20 cm away from the base of the crystallizing dish.

The mass of volatile hydrocarbon solvent of volatile linear alkane(s) type remaining in the crystallizing dish is measured at regular time intervals.

The evaporation profile of the solvent is then obtained by plotting the curve of the amount of product evaporated (in mg/cm') as a function of time (in min).

The evaporation rate, which corresponds to the tangent at the origin of the curve obtained, is then calculated. The evaporation rates are expressed in mg of volatile hydrocarbon solvent of volatile linear alkane(s) type evaporated per unit of surface area (cm²) and per unit of time (minute).

The volatile hydrocarbon solvent of volatile linear alkane(s) type according to the invention has an evaporation rate of less than or equal to 0.13 mg/cm²/min. This therefore corresponds to an amount of evaporated solvent of less than or equal to 3.9 mg/cm² in 30 minutes, preferably approximately 3.3 mg/cm² in 30 minutes.

According to one embodiment, a volatile hydrocarbon solvent of volatile linear alkane(s) type suitable for the invention may have a flashpoint included within the range of from 70 to 120° C., and more particularly from 80 to 100° C., and may especially be approximately 89° C.

According to one embodiment, a volatile hydrocarbon solvent suitable for the invention may in particular be a volatile linear alkane or a mixture of volatile linear alkanes.

In particular, a volatile hydrocarbon solvent may be a volatile linear alkane selected from volatile linear alkanes containing from 9 to 15 carbon atoms, and more particularly from 11 to 13 carbon atoms.

A volatile hydrocarbon solvent of volatile linear alkane(s) type may be a volatile linear alkane of plant origin.

Such an alkane may be obtained, directly or in several steps, from a plant starting material such as an oil, a butter, a wax, etc.

By way of example of a volatile linear alkane suitable for the invention, mention may be made of the alkanes described in patent application WO 2007/068371 from the company Cognis.

These alkanes are obtained from fatty alcohols, which are themselves obtained from coconut oil or palm oil.

By way of example of a volatile linear alkane suitable for the invention, mention may be made of n-nonane (C9), n-decane (C10), n-undecane (C11), n-dodecane (C12), n-tridecane (C13), n-tetradecane (C14), n-pentadecane (C15), and mixtures thereof, and in particular the mixture of n-undecane (C11) and n-tridecane (C13) sold under the reference Cetiol UT by the company Cognis.

More particularly, a volatile linear alkane suitable for the invention may be selected from n-nonane, n-undecane, n-dodecane, n-tridecane, and mixtures thereof.

According to one embodiment, a volatile linear alkane suitable for the invention may be used in the form of an n-undecane/n-tridecane mixture.

Preferably, the n-undecane: n-tridecane weight ratio is from 50:50 to 90:10, preferably from 60:40 to 80:20, and in particular from 65:35 to 75:25.

In particular, a composition according to the invention may comprise an n-undecane: n-tridecane mixture in a weight ratio of 70:30.

A composition of the invention may comprise from 0.5% to 90% by weight of volatile hydrocarbon solvent of volatile linear alkane(s) type, in particular from 1% to 80% by weight, and more particularly from 5% to 60% by weight of volatile hydrocarbon solvent of volatile linear alkane(s) type relative to the total weight of the composition.

The volatile hydrocarbon solvent forms, alone or with one or more other compounds listed below, the liquid fatty phase of the composition.

Physiologically Acceptable Medium

In addition to the compounds indicated above, a composition according to the invention comprises a physiologically acceptable medium.

The term “physiologically acceptable medium” is intended to denote a medium which is particularly suitable for the application of a composition of the invention to keratin fibres, in particular the hair, the eyelashes and the eyebrows.

The physiologically acceptable medium is generally suitable for the nature of the support to which the composition should be applied, and also for the way in which the composition should be packaged.

Aqueous Phase

A composition of the invention is anhydrous and may comprise water in a content of less than 10%, preferably less than 5% by weight of water, in particular less than 3%, especially less than 2%, and more particularly less than 1% by weight of water relative to the total weight of the composition.

More particularly, a composition of the invention may be devoid of water.

According to one embodiment, a composition of the invention may also comprise at least one water-miscible organic solvent.

The water-miscible organic solvent(s) suitable for the invention may be selected from C₁₋₈, and in particular C₁₋₅, monoalcohols, in particular ethanol, isopropanol, tert-butanol, n-butanol, benzyl alcohol, polyols, such as sorbitol, C₂-C₈ glycols, C₂-C₆ polyhydric alcohols, such as glycerol, C₃-C₄ ketones, C₂-C₄ aldehydes, and mixtures thereof.

Surfactants

A composition according to the invention may comprise at least one surfactant, in particular selected from amphoteric, anionic, cationic or nonionic surfactants, used alone or as a mixture.

The surfactants may be generally present in the composition in a proportion that may range, for example, from 0.3% to 30% by weight, and preferably from 0.5% to 20%, preferably from 1% to 15% by weight relative to the total weight of the composition.

According to one embodiment, a composition of the invention may comprise less than 35% by weight, or even less than 25% by weight, or even less than 20%, less than 10%, less than 5%, less than 2%, less than 1% by weight of surfactants relative to the total weight of the composition, or is even devoid of surfactants.

According to the invention, an emulsifying surfactant suitably chosen in order to obtain a water-in-oil emulsion is generally used. In particular, an emulsifying surfactant having, at 25° C., an HLB balance (hydrophilic-lipophilic balance), within the meaning of Griffin, of less than or equal to 8 may be used.

The HLB value according to Griffin is defined in J. Soc. Cosm. Chem. 1954 (volume 5), pages 249-256.

These surfactants can be selected from nonionic, anionic, cationic or amphoteric surfactants. Reference may be made to the document “Encyclopedia of Chemical Technology, Kirk-Othmer”, volume 22, p. 333-432, 3rd edition, 1979, Wiley, for the definition of the properties and emulsifying functions of surfactants, in particular p. 347-377 of this reference, for anionic, amphoteric and nonionic surfactants.

The surfactants preferably used in the composition according to the invention are selected from:

a) nonionic surfactants with an HLB of less than 8 at 25° C., optionally in combination with one or more nonionic surfactants with an HLB of greater than 8 at 25° C., such as mentioned below, such as:
esters and ethers of monosaccharides, such as sucrose stearate, sucrose cocoate, sorbitan stearate and their mixtures, for example Arlatone 2121®, sold by the company ICI, or Span 65V, from the company Uniqema;
esters of fatty acids, in particular C₈-C₂₄ and preferably C₁₆-C₂₂ fatty acids, and of a polyol, in particular of glycerol or of sorbitol, such as glyceryl stearate, for example sold under the name Tegin M® by the company Goldschmidt, glyceryl laurate, such as the product sold under the name Imwitor 312® by the company Hüls, polyglyceryl-2 stearate, sorbitan tristearate and glyceryl ricinoleate;
lecithins, such as soybean lecithins (for instance Emulmetik 100 J from Cargill, or Biophilic H from Lucas Meyer);
oxyethylenated and/or oxypropylenated ethers (which can comprise from 1 to 150 oxyethylenated and/or oxypropylenated groups) of fatty alcohols (in particular of a C₈-C₂₄ and preferably C₁₂-C₁₈ alcohol), such as the oxyethylenated ether of stearyl alcohol comprising 2 oxyethylene units (CTFA name “Steareth-2”), such as Brij 72, sold by the company Uniqema;
the cyclomethicone/dimethicone copolyol mixture sold under the name Q2-3225C® by the company Dow Corning;

b) nonionic surfactants with an HLB of greater than or equal to 8 at 25° C., used alone or as a mixture; mention may in particular be made of:
monosaccharide esters and ethers, such as the mixture of cetylstearyl glucoside and of cetyl and stearyl alcohols, for instance Montanov 68 from Seppic;
oxyethylenated and/or oxypropylenated glycerol ethers which may comprise from 1 to 150 oxyethylene and/or oxypropylene units;
oxyethylenated and/or oxypropylenated ethers (which can comprise from 1 to 150 oxyethylene and/or oxypropylene units) of fatty alcohols, in particular C₈-C₂₄ and preferably C₁₂-C₁₈ fatty alcohols, such as the oxyethylenated ether of stearyl alcohol comprising 20 oxyethylene units (CTFA name “Steareth-20”), such as Brij 78 sold by the company Uniqema, the oxyethylenated ether of cetearyl alcohol comprising 30 oxyethylene units (CTFA name “Ceteareth-30”) and the oxyethylenated ether of the mixture of C₁₂-C₁₅ fatty alcohols comprising 7 oxyethylene units (CTFA name “C₁₂₋₁₅ Pareth-7”), such as that sold under the name Neodol 25-7® by Shell Chemicals;
esters of a fatty acid, in particular a C₈-C₂₄ and preferably C₁₆-C₂₂ fatty acid, and of polyethylene glycol (or PEG) (which can comprise from 1 to 150 oxyethylene units), such as PEG-50 stearate and PEG-40 monostearate, sold under the name Myrj 52P® by the company Uniqema;
esters of a fatty acid, in particular a C₈-C₂₄ and preferably C₁₆-C₂₂ fatty acid, and of oxyethylenated and/or oxypropylenated glycerol ethers (which can comprise from 1 to 150 oxyethylene and/or oxypropylene units), such as the polyoxyethylenated glyceryl monostearate comprising 200 oxyethylene units sold under the name Simulsol 220 ™® by the company SEPPIC; polyoxyethylenated glyceryl stearate comprising 30 oxyethylene units, such as the product Tagat S® sold by the company Goldschmidt, polyoxyethylenated glyceryl oleate comprising 30 oxyethylene units, such as the product Tagat O® sold by the company Goldschmidt, polyoxyethylenated glyceryl cocoate comprising 30 oxyethylene units, such as the product Varionic LI 13® sold by the company Sherex, polyoxyethylenated glyceryl isostearate comprising 30 oxyethylene units, such as the product Tagat L® sold by the company Goldschmidt, and polyoxyethylenated glyceryl laurate comprising 30 oxyethylene units, such as the product Tagat I® from the company Goldschmidt;
esters of a fatty acid, in particular a C₈-C₂₄ and preferably C₁₆-C₂₂ fatty acid, and of oxyethylenated and/or oxypropylenated sorbitol ethers (which can comprise from 1 to 150 oxyethylene and/or oxypropylene units), such as the polysorbate 20 sold under the name Tween 20® by the company Croda, such as the polysorbate 60 sold under the name Tween 60® by the company Uniqema;
dimethicone copolyol, such as that sold under the name Q2-5220® by the company Dow Corning;
dimethicone copolyol benzoate, such as that sold under the name Finsolv SLB 10® and 201® by the company Fintex;
copolymers of propylene oxide and of ethylene oxide, also known as EO/PO polycondensates,
and mixtures thereof.

The EO/PO polycondensates are more particularly copolymers consisting of polyethylene glycol and polypropylene glycol blocks, such as, for example, polyethylene glycol/polypropylene glycol/polyethylene glycol triblock polycondensates. These triblock polycondensates have, for example, the following chemical structure:

H—(O—CH₂—CH₂)a-(O—CH(CH₃)—CH₂)b-(O—CH₂—CH₂)a-OH,

in which formula a ranges from 2 to 120 and b ranges from 1 to 100.

The EO/PO polycondensates preferably have a weight-average molecular weight ranging from 1000 to 15 000 and better still ranging from 2000 to 13 000. Advantageously, said EO/PO polycondensates have a cloud point, at 10 g/l in distilled water, of greater than or equal to 20° C., preferably of greater than or equal to 60° C. The cloud point is measured according to the ISO 1065 standard. Mention may be made, as EO/PO polycondensate which can be used according to the invention, of the polyethylene glycol/polypropylene glycol/polyethylene glycol triblock polycondensates sold under the Synperonic® names, such as Synperonic PE/L44® and Synperonic PE/F127®, by the company ICI.

c) anionic surfactants such as:
salts of C₁₆-C₃₀ fatty acids, in particular amino salts such as triethanolamine stearate or 2-amino-2-methylpropane-1,3-diol stearate;
salts of polyoxyethylenated fatty acids, in particular amino salts or alkali metal salts, and mixtures thereof;
phosphoric esters and salts thereof, such as “DEA oleth-10 phosphate” (Crodafos N 10N from the company Croda) or monopotassium monocetyl phosphate (Amphisol K from Givaudan or Arlatone MAP 160K from the company Uniqema);
sulphosuccinates, such as “disodium PEG-5 citrate lauryl sulphosuccinate” and “disodium ricinoleamido MEA sulphosuccinate”;
alkyl ether sulphates, such as sodium lauryl ether sulphate;
isethionates;
acylglutamates, such as “disodium hydrogenated tallow glutamate” (AMISOFT HS-21 R® sold by the company Ajinomoto) and sodium stearoyl glutamate (Amisoft HS-11 PF® sold by the company Ajinomoto), and mixtures thereof;
soybean derivatives, such as potassium soyate;
citrates, such as glyceryl stearate citrate (Axol C 62 Pellets from Degussa);
proline derivatives, such as sodium palmitoyl proline (Sepicalm VG from Seppic), or the mixture of sodium palmitoyl sarcosinate, magnesium palmitoyl glutamate, palmitic acid and palmitoyl proline (Sepifeel One from Seppic);
lactylates, such as sodium stearoyl lactylate (Akoline SL from Karlshamns AB);
sarcosinates, such as sodium palmitoyl sarcosinate (Nikko 1 sarcosinate PN) or the mixture of stearoyl sarcosine and myristoyl sarcosine 75/25 (Crodasin SM from Croda);
sulphonates, such as sodium C₁₄₋₁₇ alkyl-sec-sulphonate (Hostapur SAS 60 from Clariant);
glycinates, such as sodium cocoyl glycinate (Amilite GCS-12 from Ajinomoto).

By way of examples of a cationic surfactant, mention may in particular be made of:
alkylimidazolidiniums, such as isostearylethylimidonium ethosulphate,
ammonium salts, such as (C₁₂₋₃₀ alkyl)tri(C₁₋₄ alkyl)ammonium halides, for instance N,N,N-trimethyl-1-docosanaminium chloride (or behentrimonium chloride).

The compositions according to the invention may also contain one or more amphoteric surfactants, for instance N-acylamino acids, such as N-alkylaminoacetates and disodium cocoamphodiacetate, and amine oxides, such as stearamine oxide, or else silicone surfactants, for instance dimethicone copolyol phosphates, such as that sold under the name Pecosil PS100® by the company Phoenix Chemical.

According to one preferred embodiment, use is made of nonionic surfactants with an HLB of less than 8 at 25° C., and in particular of esters of fatty acids, in particular C₈-C₂₄ and preferably C₁₆-C₂₂ fatty acids, and of a polyol, in particular of glycerol or of sorbitol. More particularly use is made as nonionic surfactant of glyceryl stearate.

Hydrophilic Thickeners

Depending on the fluidity of the composition that it is desired to obtain, one or more hydrophilic thickeners may be incorporated into a composition of the invention.

The term “hydrophilic thickener or gelling agent” is intended to mean a gelling agent or thickener that is water-soluble or water-dispersible.

As hydrophilic gelling agents, mention may in particular be made of water-soluble or water-dispersible thickening polymers. Said polymers may in particular be selected from:
modified or unmodified carboxyvinyl polymers, such as the products sold under the name Carbopol (CTFA name: carbomer) by the company Goodrich;
homo- or copolymers of acrylic or methacrylic acids and salts thereof or esters thereof, in particular the products sold under the names Versicol F® or Versicol K® by the company Allied Colloid, Utrahold 8® by the company Ciba-Geigy, polyacrylates and polymethacrylates, such as the products sold under the names Lubrajel and Norgel by the company Guardian or under the name Hispagel by the company Hispano Chimica, polyacrylic acids of Synthalen K type;
polyacrylamides; acrylic acid/acrylamide copolymers sold in the form of their sodium salt, under the name Reten® by the company Hercules, the poly(sodium methacrylate) sold under the name Darvan No. 7® by the company Vanderbilt, the sodium salts of polyhydroxycarboxylic acids sold under the name Hydagen F® by the company Henkel;
2-acrylamido-2-methylpropanesulphonic acid polymers and copolymers, which are optionally crosslinked and/or neutralized, such as the poly(2-acrylamido-2-methylpropanesulphonic acid) sold by the company Clariant under the name “Hostacerin AMPS” (CTFA name: ammonium polyacryldimethyltauramide);
crosslinked anionic acrylamide/AMPS copolymers, in the form of a W/O emulsion, such as those sold under the name Sepigel 305 (CTFA name: polyacrylamide/C₁₃₋₁₄ isoparaffin/laureth-7) and under the name Simulgel 600 (CTFA name: acrylamide/sodium acryloyldimethyltaurate copolymer/isohexadecane/polysorbate 80) by the company SEPPIC;
polyacrylic acid/alkyl acrylate copolymers of Pemulen type;
polysaccharide biopolymers, such as xanthan gum, guar gum, carob gum, gum acacia, scleroglucans, chitin derivatives and chitosan derivatives, carrageenans, gellans, alginates, or celluloses such as microcrystalline cellulose, carboxymethylcellulose, hydroxymethylcellulose, hydroxyethylcellulose and hydroxypropylcellulose;
hydrophilic clays, hydrophilic fumed silica;
and mixtures thereof.

The term “hydrophilic clay” is intended to mean a clay capable of swelling in water; this clay swells in water and forms, after hydration, a colloidal dispersion.

The clays are products already well known per se, which are described, for example, in the book “Minéralogie des argiles [Clay mineralogy], S. Caillère, S. Hénin, M. Rautureau, 2nd edition 1982, Masson”. The clays are silicates containing a cation that can be selected from calcium, magnesium, aluminium, sodium, potassium and lithium cations, and mixtures thereof. By way of example of such products, mention may be made of the clays of the smectite family, such as montmorillonites, hectorites, bentonites, beidellites or saponites, and also of the vermiculite family, the stevensite family and the chlorite family.

These clays may be of natural or synthetic origin.

As a hydrophilic clay, mention may be made of smectites such as saponites, hectorites, montmorillonites, bentonites and beidellite.

As a hydrophilic clay, mention may be made of synthetic hectorites (also known as laponites), such as the products sold by Laporte under the name Laponite XLG, Laponite RD and Laponite RDS (these products are sodium magnesium silicates, and in particular sodium magnesium lithium silicates); bentonites, such as the product sold under the name Bentone HC by the company Rheox; magnesium aluminium silicates, in particular hydrates, such as the product sold by the company Vanderbilt Company under the name Veegum ultra, Veegum HS and Veegum DGT, or else calcium silicates, and in particular that in synthetic form sold by the company under the name Micro-cel C.

The hydrophilic fumed silicas can be obtained by high-temperature hydrolysis of a volatile silicon compound in an oxhydric flame, producing a finely divided silica. Hydrophilic silicas have a large number of silanol groups at their surfaces. Such hydrophilic silicas are, for example, sold under the names “Aerosil 130®, “Aerosil 200®”, “Aerosil 255®”, “Aerosil 300®”, and “Aerosil 380®” by Degussa, and “Cab-O-Sil HS5®”, “Cab-O-Sil EH-5®”, “Cab-O-Sil LM-130®”, “Cab-O-Sil MS-55®” and “Cab-O-Sil M-5®” by the company Cabot.

The hydrophilic fumed silica preferably has a particle size that may be nanometric to micrometric, for example ranging from approximately 5 to 200 nm.

According to one embodiment, a composition of the invention may comprise from 0.01% to 30% by weight of hydrophilic thickeners, in particular from 0.5% to 20% by weight, and more particularly from 1% to 15% by weight of hydrophilic thickeners relative to the total weight of the composition.

Liquid Fatty Phase

A cosmetic composition according to the present invention may comprise, in addition to the volatile hydrocarbon solvent of C₉-C₁₅ volatile linear alkane(s) type mentioned above, at least one additional liquid and/or solid fatty phase and in particular at least one oil as mentioned hereinafter.

The term “oil” is intended to mean any fatty substance in liquid form at ambient temperature (20-25° C.) and at atmospheric pressure.

A composition of the invention may comprise an additional liquid fatty phase in a content ranging from 1% to 50%, in particular from 5% to 40%, in particular from 10% to 30%, and more particularly from 15% to 20% by weight relative to the total weight of the composition.

According to one embodiment, a composition of the invention is devoid of additional liquid fatty phase.

The liquid fatty phase or oily phase suitable for the preparation of the cosmetic compositions according to the invention may comprise hydrocarbon-based oils, hydrocarbon oils, silicone, fluoro or non-fluoro oils, or mixtures thereof.

The oils may be volatile or non-volatile.

They may be of animal, plant, mineral or synthetic origin.

For the purpose of the present invention, the term “volatile oil” is intended to mean an oil (or nonaqueous medium) capable of evaporating on contact with the skin in less than one hour, at ambient temperature and at atmospheric pressure. The volatile oil is a volatile cosmetic oil which is liquid at ambient temperature, having in particular a non-zero vapour pressure, at ambient temperature and at atmospheric pressure, in particular having a vapour pressure ranging from 0.13 Pa to 40 000 Pa (10⁻³ to 300 mmHg), and preferably ranging from 1.3 Pa to 13 000 Pa (0.01 to 100 mmHg), and preferentially ranging from 1.3 Pa to 1300 Pa (0.01 to 10 mmHg).

For the purpose of the present invention, the term “non-volatile oil” is intended to mean an oil having a vapour pressure of less than 0.13 Pa.

For the purpose of the present invention, the term “silicone oil” is intended to mean an oil comprising at least one silicon atom, and in particular at least one Si—O group.

The term “fluoro oil” is intended to mean an oil comprising at least one fluorine atom.

The term “hydrocarbon oil” is intended to mean an oil containing mainly hydrogen and carbon atoms, and if necessary alcohol, acid, ester or ether functions.

A hydrocarbon-based oil is exclusively composed of hydrogen and carbon atoms.

The oils may optionally comprise oxygen, nitrogen, sulphur and/or phosphorus atoms, for example in the form of hydroxyl or acid radicals.

An oil may be a polymeric or non-polymeric oil. The expression “polymeric oil” is understood to mean an oil comprising at least one or being exclusively composed of molecules composed of repeting monomers, identical or different. As polymeric oil, mention may be made of polyisobutene.

Volatile Oils

The volatile oils may be selected from hydrocarbon-based oils containing from 8 to 16 carbon atoms, and in particular branched C₈-C₁₆ alkanes (also known as isoparaffins), such as isododecane (also known as 2,2,4,4,6-pentamethylheptane), isodecane or isohexadecane, and for example the oils sold under the trade names Isopars® or Permethyls®.

Advantageously, a composition of the invention comprises less than 5%, or even less than 2% by weight of isododecane relative to the total weight of the composition, or is even devoid of isododecane.

Use may also be made, as volatile oils, of volatile silicones, such as, for example, volatile linear or cyclic silicone oils, in particular those having a viscosity ≦8 centistokes (cSt) (8×10⁻⁶ m²/s) and having in particular from 2 to 10 silicon atoms, and in particular from 2 to 7 silicon atoms, these silicones optionally comprising alkyl or alkoxy groups having from 1 to 10 carbon atoms. As volatile silicone oil that can be used in the invention, mention may in particular be made of dimethicones having a viscosity of 5 and 6 cSt, octamethylcyclotetrasiloxane, decamethylcyclopentasiloxane, dodecamethylcyclo-hexasiloxane, heptamethylhexyltrisiloxane, heptamethyloctyltrisiloxane, hexamethyl-disiloxane, octamethyltrisiloxane, decamethyltetrasiloxane, dodecamethylpentasiloxane, and mixtures thereof.

Use may also be made of volatile fluoro oils, such as nonafluoromethoxybutane or perfluoromethylcyclopentane, and mixtures thereof.

According to one embodiment, a composition of the invention may advantageously comprise less than 10% by weight, or even less than 5% by weight, or even less than 2% by weight relative to the total weight of the composition, or may even be devoid of cyclic silicone oil.

Non-Volatile Oils

The non-volatile oils may in particular be selected from non-volatile hydrocarbon, fluorinated and/or silicone oils.

As non-volatile hydrocarbon oil, mention may in particular be made of:
hydrocarbon oils of animal origin,
hydrocarbon oils of plant origin, such as phytostearyl esters, for instance phytostearyl oleate, phytostearate isostearate and lauroyl/octyldodecyl/phytostearyl glutamate (Ajinomoto, Eldew PS203), triglycerides constituted of fatty acid esters of glycerol, in particular in which the fatty acids may have chain lengths ranging from C₄ to C₃₆, and in particular from C₁₈ to C₃₆, it being possible for these oils to be linear or branched, and saturated or unsaturated; these oils may in particular be heptanoic or octanoic triglycerides, shea oil, alfalfa oil, poppyseed oil, pumpkin oil, millet oil, barley oil, quinoa oil, rye oil, candlenut oil, passion flower oil, shea butter, aloe oil, sweet almond oil, peach kernel oil, groundnut oil, argan oil, avocado oil, baobab oil, borage oil, broccoli oil, calendula oil, camelina oil, carrot oil, safflower oil, hemp oil, rapeseed oil, cottonseed oil, coconut oil, marrow seed oil, wheat germ oil, jojoba oil, lily oil, macadamia oil, maize oil, meadowfoam oil, St. John's wort oil, monoi oil, hazelnut oil, apricot kernel oil, nut oil, olive oil, evening primrose oil, palm oil, blackcurrant seed oil, kiwi seed oil, grapeseed oil, pistachio oil, pumpkin oil, winter squash oil, quinoa oil, musk rose oil, sesame oil, soya oil, sunflower oil, castor oil and watermelon oil, and mixtures thereof, or alternatively caprylic/capric acid triglycerides, for instance those sold by the company Stearineries Dubois or those sold under the names Miglyol 810®, 812® and 818® by the company Dynamit Nobel,
synthetic ethers containing from 10 to 40 carbon atoms;
synthetic esters, for instance oils of formula R₁COOR₂, in which R₁ represents a linear or branched fatty acid residue containing from 1 to 40 carbon atoms and R₂ represents a hydrocarbon chain, in particular a branched chain, containing from 1 to 40 carbon atoms provided that R₁+R₂ is 10. The esters may in particular be selected from fatty acid alcohol and esters, such as, for example:
   cetostearyl octanoate, isopropyl alcohol esters, such as isopropyl myristate or isopropyl palmitate, ethyl palmitate, 2-ethylhexyl palmitate, isopropyl stearate or isostearate, isostearyl isostearate, octyl stearate, hydroxylated esters, for instance isostearyl lactate, octyl hydroxystearate, diisopropyl adipate, heptanoates, and especially isostearyl heptanoate, alcohol or polyalcohol octanoates, decanoates or ricinoleates, for instance propylene glycol dioctanoate, cetyl octanoate, tridecyl octanoate, 2-ethyl-hexyl 4-diheptanoate and palmitate, alkyl benzoate, polyethylene glycol diheptanoate, propylene glycol 2-diethylhexanoate, and mixtures thereof, C₁₂-C₁₅ alkyl benzoates, hexyl laurate, neopentanoic acid esters, for instance isodecyl neopentanoate, isotridecyl neopentanoate, isostearyl neopentanoate or octyldodecyl neopentanoate, isononanoic acid esters, for instance isononyl isononanoate, isotridecyl isononanoate and octyl isononanoate, hydroxylated esters such as isostearyl lactate and diisostearyl malate;
polyol esters and pentaerythritol esters, for instance dipentaerythrityl tetrahydroxy-stearate/tetraisostearate,
esters of diol dimers and diacid dimers, such as Lusplan DD-DA5® and Lusplan DD-DA7®, sold by the company Nippon Fine Chemical and described in application US 2004-175338,
copolymers of a diol dimer and of a diacid dimer, and esters thereof, such as copolymers of dilinoleyl diol dimers/dilinoleic dimers and esters thereof, for instance Plandool-G,
copolymers of polyols and of diacid dimers, and esters thereof, such as Hailuscent ISDA,
fatty alcohols that are liquid at ambient temperature, with a branched and/or unsaturated carbon chain containing from 12 to 26 carbon atoms, for instance 2-octyldodecanol, isostearyl alcohol, oleyl alcohol, 2-hexyldecanol, 2-butyloctanol and 2-undecylpentadecanol,
C₁₂-C₂₂ higher fatty acids, such as oleic acid, linoleic acid or linolenic acid, and mixtures thereof, and
dialkyl carbonates, the two alkyl chains possibly being identical or different, such as dicaprylyl carbonate sold under the name Cetiol CC® by Cognis,
oils of high molar mass, having in particular a molar mass ranging from approximately 400 to approximately 10 000 g/mol, in particular from approximately 650 to approximately 10 000 g/mol, in particular from approximately 750 to approximately 7500 g/mol, and more particularly ranging from approximately 1000 to approximately 5000 g/mol. As oils of high molar mass that can be used in the present invention, mention may in particular be made of the oils selected from:
   lipophilic polymers,
   linear fatty acid esters having a total carbon number ranging from 35 to 70,
   hydroxylated esters,
   aromatic esters,
   esters of C₂₄-C₂₈ branched fatty acids or fatty alcohols,
   silicone oils,
   oils of plant origin,
   and mixtures thereof.

For example, an oil of high molar mass may be selected from:
a) lipophilic polymers, such as:
   polybutylenes, such as Indopol H-100 (of molar mass MM=965 g/mol), Indopol H-300 (MM=1340 g/mol), Indopol H-1500 (MM=2160 g/mol) sold or manufactured by the company Amoco,
   polyisobutylenes, for example hydrogenated polyisobutylenes, such as Panalane H-300 E sold or manufactured by the company Amoco (MM=1340 g/mol), Viseal 20000 sold or manufactured by the company Synteal (MM=6000 g/mol), Rewopal PIB 1000 sold or manufactured by the company Witco (MM=1000 g/mol),
   polydecenes and hydrogenated polydecenes, such as: Puresyn 10 (MM=723 g/mol), Puresyn 150 (MM=9200 g/mol) sold or manufactured by the company Mobil Chemicals,
   vinylpyrrolidone copolymers, such as: the vinylpyrrolidone/1-hexadecene copolymer Antaron V-216 sold or manufactured by the company ISP (MM=7300 g/mol), and polyvinylpyrrolidone (PVP) copolymers, such as copolymers of a C₂-C₃₀, for instance C₃-C₂₂, alkene, and combinations thereof, may be used. As examples of PVP copolymers that may be used in the invention, mention may be made of the PVP/vinyl laurate copolymer, the PVP/vinyl stearate copolymer, butylated PVP, the PVP/hexadecene copolymer, the PVP/triacontene copolymer or the PVP/acrylic acid/lauryl methacrylate copolymer,
b) esters, such as:
   esters of linear fatty acids having a total carbon number ranging from 35 to 70, such as pentaerythrityl tetrapelargonate (MM=697 g/mol),
   hydroxylated esters, such as polyglyceryl-2 triisostearate (MM=965 g/mol),
   aromatic esters, such as tridecyl trimellitate (MM=757 g/mol),
   esters of C₂₄-C₂₈ branched fatty acids or fatty alcohols, such as those described in U.S. Pat. No. 6,491,927, and pentaerythritol esters, and especially triisoarachidyl citrate (MM=1033.76 g/mol), pentaerythrityl tetraisononanoate (MM=697 g/mol), glyceryl triisostearate (MM=891 g/mol), glyceryl 2-tridecyl tetradecanoate (MM=1143 g/mol), pentaerythrityl tetraisostearate (MM=1202 g/mol), polyglyceryl-2 tetraisostearate (MM=1232 g/mol) or else pentaerythrityl 2-tetradecyl tetradecanoate (MM=1538 g/mol),
   diol dimer esters and polyesters, such as the diol dimer esters of a fatty acid and the diol dimer esters of a diacid, for instance Lusplan DD-DA5® and Lusplan DD-DA7® sold by the company Nippon Fine Chemical and described in application US 2004-175338,
c) silicone oils, such as phenyl silicones, for instance Belsil PDM 1000 from the company Wacker (MM=9000 g/mol). Other non-volatile silicone oils that can be used in the composition according to the invention may be non-volatile polydimethylsiloxanes (PDMS), PDMSs comprising alkyl or alkoxy groups that are pendent and/or at the end of a silicone chain, these groups each containing from 2 to 24 carbon atoms, phenyl silicones, for instance phenyl trimethicones, phenyl dimethicones, phenyltrimethyl-siloxydiphenylsiloxanes, diphenyl dimethicones, diphenylmethyldiphenyl-trisiloxanes and 2-phenylethyl trimethylsiloxysilicates, and dimethicones or phenyl trimethicone with a viscosity of less than or equal to 100 cSt, and mixtures thereof,
and also mixtures of the oils a) and/or b) and/or c).

According to one embodiment, a composition of the invention may comprise less than 25% by weight, less than 20%, or even less than 10%, less than 5%, less than 2%, less than 1% by weight of non-volatile oil, relative to the total weight of the composition, or is even devoid of non-volatile oil.

In another embodiment, a composition of the invention may contain at least 20% by weight of volatile hydrocarbon solvent of volatile linear alkane(s) type relative to the total weight of hydrocarbon oil of the composition.

In one embodiment a composition of the invention may contain at least 30%, or even at least 40%, in particular at least 50%, notably at least 60%, more particularly at least 70% and more particularly at least 80%, at least 90% or 100% of volatile hydrocarbon solvent of volatile linear alkane(s) type relative to the total weight of hydrocarbon oil of the composition.

A composition according to the invention containing 100% of volatile hydrocarbon solvent of volatile linear alkane(s) type relative to the total weight of hydrocarbon oil comprise a hydrocarbon oil phase composed exclusively of volatile hydrocarbon solvent of volatile linear alkane(s) type.

In one variant embodiment, a composition according to the invention may contain at least 20% of volatile hydrocarbon solvent of volatile linear alkane(s) type relative to the total weight of hydrocarbon-based oil, in particular relative to the total weight of saturated hydrocarbon-based oil, and preferably relative to the total weight of non-polymeric saturated hydrocarbon-based oil of the composition.

In one embodiment a composition of the invention may contain at least 30%, or even at least 40%, in particular at least 50%, notably at least 60%, more particularly at least 70%, and more particularly at least 80%, at least 90% or 100% of volatile hydrocarbon solvent of volatile linear alkane(s) type relative to the total weight of hydrocarbon-based oil, in particular relative to the total weight of saturated hydrocarbon-based oil, and preferably relative to the total weight of non-polymeric saturated hydrocarbon-based oil of the composition.

In one variant embodiment, a hydrocarbon-based oil considered in the previous embodiments may present a molar mass below 650 g/mol, and in particular below 400 g/mol.

A composition of the invention containing 100% of volatile hydrocarbon solvent of volatile linear alkane(s) type relative to the total weight of hydrocarbon-based oil or of saturated hydrocarbon-based oil, or of non-polymeric saturated hydrocarbon-based oil, comprise a hydrocarbon-based oil, saturated hydrocarbon-based oil or of non-polymeric saturated hydrocarbon-based oil phase exclusively composed of volatile hydrocarbon solvent of volatile linear alkane(s) type.

Lipophilic Structuring Agent

A composition according to the invention may comprise at least one agent for structuring a liquid fatty phase, selected from waxes, preferably polar waxes, semicrystalline polymers, lipophilic gelling agents, pasty compounds, and mixtures thereof.

According to one particular embodiment, the structuring agent is selected from waxes, preferably polar waxes, lipophilic gelling agents, and mixtures thereof.

A composition according to the invention may comprise a content of agent for structuring a liquid fatty phase ranging from 1% to 50% by weight relative to the total weight of the composition; it may in particular contain from 5% to 45%, more particularly from 10% to 40%.

Wax(es)

A composition of the invention may comprise at least one wax. A wax suitable for the invention is, in general, a lipophilic compound which is solid at ambient temperature (25° C.), which undergoes a reversible solid/liquid change of state, and which has a melting point of greater than or equal to 30° C., possibly ranging up to 200° C., and especially up to 120° C.

On bringing a wax to the liquid state (melting), it is possible to render it miscible with oils and to form a macroscopically homogenous mixture, but on bringing the temperature of the mixture back to ambient temperature, recrystallization of the wax in the oils of the mixture is obtained.

A wax suitable for the invention may have a melting point of greater than or equal to 45° C., and in particular greater than or equal to 55° C.

For the purpose of the invention, the melting point corresponds to the temperature of the most endothermic peak observed by thermal analysis (DSC), as described in ISO standard 11357-3; 1999. The melting point of the wax can be measured using a differential scanning calorimeter (DSC), for example the calorimeter sold under the name “MDSC 2920” by the company TA Instruments.

According to a preferred embodiment, a polar wax is used.

For the purpose of the present invention, the term “polar wax” is intended to mean a wax of which the solubility parameter at 25° C., δₐ, is other than 0 (J/cm³)^{1/2}.

In particular, the term “polar” wax is intended to mean a wax in which the chemical structure is essentially formed, or even constituted, of carbon and hydrogen atoms, and comprises at least one highly electronegative heteroatom such as an oxygen, nitrogen or phosphorus atom.

The definition and the calculation of the solubility parameters in the Hansen three-dimensional solubility space are described in the article by C. M. Hansen: “The three dimensional solubility parameters” J. Paint Technol. 39, 105 (1967).

According to this Hansen space:
δ_{D} characterizes the London dispersion forces derived from the formation of dipoles induced during molecular impacts;
δ_{P} characterizes the Debye interaction forces between permanent dipoles and also the Keesom interaction forces between induced dipoles and permanent dipoles;
δₕ characterizes the specific interaction forces (such as hydrogen bonding, acid/base, donor/acceptor, etc.);
δₐ is determined by the equation: δₐ=(δₚ²+δₕ²)^{1/2}.

The parameters δₚ, δₕ, δ_{D} and δₐ are expressed in (J/cm³)^{1/2}.

The polar waxes that can be used in the compositions according to the invention are selected from waxes, which are solid at ambient temperature, of animal, plant and/or mineral origin and/or which are synthetic, and mixtures thereof.

The waxes may in particular be hydrocarbon waxes or silicone waxes.

The term “silicone wax” is intended to mean an oil comprising at least one silicon atom, and in particular comprising Si—O groups.

The term “hydrocarbon wax” is intended to mean a wax formed essentially, or even constituted, of carbon and hydrogen atoms and, optionally, oxygen and/or nitrogen atoms, and containing no silicon or fluorine atom. It may contain alcohol, ester, ether, carboxylic acid, amine and/or amide groups.

In particular, a wax suitable for the invention may be selected from waxes of animal, plant, mineral or synthetic origin, and mixtures thereof.

By way of illustration of waxes suitable for the invention, mention may in particular be made of hydrocarbon waxes, such as beeswax, especially of biological origin, lanolin wax and Chinese insect waxes; rice bran wax, carnauba wax, candelilla wax, ouricury wax, esparto wax, berry wax, shellac wax, Japan wax and sumac wax; montan wax, orange and lemon waxes, microcrystalline waxes, paraffins and ozokerite; polyethylene waxes, waxes obtained by Fischer-Tropsch synthesis and waxy copolymers, and esters thereof.

Mention may also be made of C₂₀-C₆₀ microcrystalline waxes, such as Microwax HW.

Mention may also be made of the MW 500 polyethylene wax sold under the reference Permalen 50-L polyethylene.

Mention may also be made of waxes obtained by catalytic hydrogenation of animal or plant oils having linear or branched C₈-C₃₂ fatty chains.

Among these, mention may in particular be made of isomerized jojoba oil, such as the transisomerized partially hydrogenated jojoba oil manufactured or sold by the company Desert Whale under the commercial reference Iso-Jojoba-50®, hydrogenated sunflower oil, hydrogenated castor oil, hydrogenated coconut oil, hydrogenated lanolin oil and di(1,1,1-trimethylolpropane) tetrastearate sold under the name Hest 2T-4S® by the company Heterene.

Mention may also be made of silicone waxes (C₃₀₋₄₅ alkyl dimethicone) and fluorinated waxes.

Use may also be made of the waxes obtained by hydrogenation of castor oil esterified with cetyl alcohol, which are sold under the names Phytowax ricin 16L64® and 22L73® by the company Sophim. Such waxes are described in application FR-A-2792190.

As wax, use may be made of a C₂₀-C₄₀ alkyl (hydroxystearyloxy)stearate (the alkyl group containing from 20 to 40 carbon atoms), alone or as a mixture.

Such a wax is in particular sold under the names Kester Wax K 82 P®, Hydroxypolyester K 82 P® and Kester Wax K 80 P® by the company Koster Keunen.

As microwaxes that may be used in a composition of the invention, mention may in particular be made of carnauba microwaxes, such as the product sold under the name MicroCare 350® by the company Micro Powders, synthetic-wax microwaxes, such as the product sold under the name MicroEase 1145® by the company Micro Powders, microwaxes constituted of a mixture of carnauba wax and of polyethylene wax, such as those sold under the names Micro Care 300® and 310® by the company Micro Powders, microwaxes constituted of a mixture of carnauba wax and of synthetic wax, such as the product sold under the name Micro Care 325® by the company Micro Powders, polyethylene microwaxes, such as those sold under the names Micropoly 200®, 220®, 220L® and 2505® by the company Micro Powders and polytetrafluoroethylene microwaxes, such as those sold under the names Microslip 519® and 519 L® by the company Micro Powders.

According to one embodiment, a wax suitable for the invention may in particular be selected from candelilla wax, carnauba wax, rice bran wax, beeswax, in particular certified biological beeswax, isomerized jojoba oil, paraffin wax, microcrystalline wax and mixtures thereof.

According to one preferred embodiment, the wax is selected from carnauba wax, paraffin wax, microcrystalline waxes and mixtures thereof.

According to one embodiment, a composition of the invention may comprise from 1% to 40% by weight of wax, in particular from 8% to 30%, and more particularly from 5% to 20% by weight of wax relative to the total weight of the composition.

Pasty Compounds

A composition according to the invention may comprise at least one pasty compound.

For the purpose of the present invention, the term “pasty” is intended to mean a lipophilic fatty compound which undergoes a reversible solid/liquid change of state and which comprises, at a temperature of 23° C., a liquid fraction and a solid fraction.

In other words, the starting melting point of the pasty compound is less than 23° C. The liquid fraction of the pasty compound measured at 23° C. represents from 23% to 97% by weight of the compound. This liquid fraction at 23° C. preferably represents between 40% and 85% by weight of the compound.

The liquid fraction by weight of the pasty compound at 23° C. is equal to the ratio of the enthalpy of fusion consumed at 23° C. to the enthalpy of fusion of the pasty compound.

The enthalpy of fusion of the pasty compound is the enthalpy consumed by the compound to change from the solid state to the liquid state. The pasty compound is “in the solid state” when the whole of its mass is in the solid form. The pasty compound is “in the liquid state” when the whole of its mass is in the liquid form.

The enthalpy of fusion of the pasty compound is equal to the area under the curve of the thermogram obtained using a differential scanning calorimeter (DSC), such as the calorimeter sold under the name MDSC 2920 by the company TA Instruments, with a rise in temperature of 5 or 10° C. per minute, according to ISO standard 11357-3; 1999. The enthalpy of fusion of the pasty compound is the amount of energy necessary to change the compound from the solid state to the liquid state. It is expressed in J/g.

The enthalpy of fusion consumed at 23° C. is the amount of energy absorbed by the sample to change from the solid state to the state which it exhibits at 23° C., composed of a liquid fraction and of a solid fraction.

The liquid fraction of the pasty compound measured at 32° C. preferably represents from 40% to 100% by weight of the compound, preferably from 50% to 100%, preferably 80% to 100%, more preferably from 90% to 100% by weight of the compound. When the liquid fraction of the pasty compound measured at 32° C. is equal to 100%, the temperature of the end of the melting range of the pasty compound is less than or equal to 32° C.

The liquid fraction of the pasty compound measured at 32° C. is equal to the ratio of the enthalpy of fusion consumed at 32° C. to the enthalpy of fusion of the pasty compound. The enthalpy of fusion consumed at 32° C. is calculated in the same way as the enthalpy of fusion consumed at 23° C.

The pasty compound is preferably selected from synthetic compounds and compounds of plant origin. A pasty compound can be obtained by synthesis from starting products of plant origin.

The presence of a pasty compound may make it possible to advantageously confer improved comfort when a composition of the invention is deposited on keratin fibres.

Such a compound may advantageously be selected from:
lanolin and derivatives thereof,
polymeric or nonpolymeric silicone compounds,
polymeric or nonpolymeric fluorinated compounds,
vinyl polymers, in particular:
   olefin homopolymers,
   olefin copolymers,
   hydrogenated diene homopolymers and copolymers,
   linear or branched and homo- or copolymeric oligomers of alkyl (meth)acrylates preferably having a C₈-C₃₀ alkyl group,
   homo- and copolymeric oligomers of vinyl esters having C₈-C₃₀ alkyl groups,
   homo- and copolymeric oligomers of vinyl ethers having C₈-C₃₀ alkyl groups,
liposoluble polyethers resulting from polyetherification between one or more C₂-C₁₀₀, in particular C₂-C₅₀, diols,
fatty acid or alcohol esters,
and mixtures thereof.

Among the esters, mention may in particular be made of:
the esters of an oligomeric glycerol, especially the esters of diglycerol, in particular the condensates of adipic acid and of glycerol, for which a portion of the hydroxyl groups of the glycerols have reacted with a mixture of fatty acids, such as stearic acid, capric acid, stearic acid and isostearic acid and 12-hydroxystearic acid, such as in particular those sold under the Softisan 649 brand by the company Sasol, or such as bis-diglyceryl polyacyladipate-2,
the arachidyl propionate sold under the Waxenol 801 brand by Alzo,
phytosterol esters,
triglycerides of fatty acids and derivatives thereof, such as hydrogenated cocoglycerides,
noncrosslinked polyesters resulting from the polycondensation between a linear or branched C₄-C₅₀ dicarboxylic acid or polycarboxylic acid and a C₂-C₅₀ diol or polyol,
aliphatic esters of an ester resulting from the esterification of an aliphatic hydroxycarboxylic acid ester with an aliphatic carboxylic acid, (Salacos HClS (V)-L sold by the company Nishing Oil),
polyesters resulting from the esterification, with a polycarboxylic acid, of an aliphatic hydroxycarboxylic acid ester, said ester comprising at least two hydroxyl groups, such as the products Risocast DA-H® and Risocast DA-L®,
and mixtures thereof.

The pasty compound may also be selected from compounds of plant origin.

Among these, mention may in particular be made of orange wax, for instance that which is sold under the reference Orange Peel Wax by the company Koster Keunen, shea butter, partially hydrogenated olive oil, for instance the compound sold under the reference Beurrolive by the company Soliance, or else cocoa butter.

The pasty compounds may be used in an amount ranging from 1% to 50% by weight, in particular from 3% to 45%, and more particularly from 5% to 40% by weight relative to the total weight of the composition.

Lipophilic Gelling Agents

The composition may comprise a lipophilic gelling agent or thickener. A gelling agent or lipophilic thickener may be inorganic or organic.

As inorganic lipophilic gelling agents, mention may, for example, be made of modified clays, such as modified magnesium silicate (Bentone gel VS38 from Rheox), or the hectorite modified with distearyldimethylammonium chloride (CTFA name: disteardimonium hectorite) sold under the name “Bentone 38 CE” by the company Rheox.

As inorganic lipophilic gelling agent, mention may also be made of optionally modified clays, for instance hectorites modified with a C₁₀ to C₂₂ fatty acid ammonium chloride, for instance hectorite modified with distearyldimethylammonium chloride, such as, for example, that sold under the name Bentone 38V® by the company Elementis.

Mention may also be made of fumed silica, optionally with a hydrophobic surface treatment, where the particle size is less than 1 μm. It is indeed possible to carry out chemical modification of the surface of the silica, by a chemical reaction producing a decrease in the number of silanol groups present at the surface of the silica. It is in particular possible to substitute silanol groups with hydrophobic groups: the resulting silica is then hydrophobic. The hydrophobic groups may be:
trimethylsiloxyl groups, which are in particular obtained by treating fumed silica in the presence of hexamethyldisilazane. Silicas treated in this way are called “silica silylate” according to the CTFA (6th Edition, 1995). They are, for example, sold under the references Aerosil R812® by the company Degussa and Cab-O-Sil TS-530® by the company Cabot,
dimethylsilyloxyl or polydimethylsiloxane groups, which are in particular obtained by treatment of fumed silica in the presence of polydimethylsiloxane or of dimethyldichlorosilane. Silicas treated in this way are called “silica dimethyl silylate” according to the CTFA (6th Edition, 1995). They are, for example, sold under the references Aerosil R972® and Aerosil R974® by the company Degussa and Cas-O-Sil TS-610® and Cab-O-Sil TS-720® by the company Cabot.

The hydrophobic fumed silica has in particular a particle size which may be nanometric to micrometric, for example ranging approximately from 5 to 200 nm.

An organic lipophilic gelling agent may be selected from polymeric organic lipophilic gelling agents which are, for example:
partially or totally crosslinked elastomeric organopolysiloxanes with a three-dimensional structure, such as those sold under the names KSG6®, KSG16® and KSG18® by the company Shin-Etsu, Trefil E-505C® and Trefil E-506C® by the company Dow-Corning, Gransil SR-CYC®, SR DMF10®, SR-DC556®, SR 5CYC Gel®, SR DMF 10 Gel® and SR DC 556 Gel® by the company Grant Industries, and SF 1204® and JK 113® by the company General Electric;
ethylcellulose, such as that sold under the name Ethocel® by the company Dow Chemical;
polyamide-type polycondensates resulting from condensation between (α) at least one acid selected from dicarboxylic acids containing at least 32 carbon atoms, such as dimeric fatty acids, and (β) an alkylenediamine, in particular ethylenediamine, in which the polyamide polymer comprises at least one terminal carboxylic acid group which is esterified or amidified with at least one monoalcohol or one monoamine containing from 12 to 30 carbon atoms, and linear and saturated, and in particular ethylenediamine/stearyl dilinoleate copolymers such as that sold under the name Uniclear 100 VG® by the company Arizona Chemical;
silicone-comprising polyamides of the polyorganosiloxane type, such as those described in documents U.S. Pat. No. 5,874,069, U.S. Pat. No. 5,919,441, U.S. Pat. No. 6,051,216 and U.S. Pat. No. 5,981,680, for instance those sold under the reference Dow Corning 2-8179 Gellant by the company Dow Corning;
galactomannans containing from one to six, and in particular from two to four, hydroxyl groups per monosaccharide, substituted with a saturated or unsaturated alkyl chain, such as guar gum alkylated with C₁ to C₆ and more particularly C₁ to C₃ alkyl chains, and mixtures thereof;
block copolymers of “diblock”, “triblock” or “radial” type, of the polystyrene/polyisoprene or polystyrene/polybutadiene type, such as those sold under the name Luvitol HSB® by the company BASF, of the polystyrene/copoly(ethylene-propylene) type, such as those sold under the name Kraton® by the company Shell Chemical Co, or else of the polystyrene/copoly(ethylene-butylene) type, blends of triblock and radial (star) copolymers in isododecane, such as those sold by the company Penreco under the name VersaGel®, for instance the mixture of butylene/ethylene/styrene triblock copolymer and of ethylene/propylene/styrene star copolymer in isododecane (Versagel M 5960).

Among the lipophilic gelling agents that may be used in a cosmetic composition of the invention, mention may also be made of esters of dextrin and of a fatty acid, such as dextrin palmitates, in particular such as those sold under the name Rheopearl TL® or Rheopearl KL® by the company Chiba Flour.

By way of organic lipophilic gelling agents suitable for the invention, mention may also be made of hydrogenated plant oils, such as hydrogenated castor oil.

By way of organic lipophilic gelling agent also suitable for the invention, mention may be made of fatty alcohols, in particular C₈ to C₂₆ fatty alcohols, and more particularly C₁₂ to C₂₂ fatty alcohols.

According to one embodiment, a fatty alcohol suitable for the invention may be selected from myristyl alcohol, cetyl alcohol, stearyl alcohol and behenyl alcohol.

By way of lipophilic gelling agent also suitable for the invention, mention may be made of fatty acid esters of glycerols, such as glyceryl stearate.

According to one embodiment, a composition of the invention may comprise at least one lipophilic gelling agent, in particular selected from inorganic lipophilic gelling agents such as modified clays, for instance bentones, or fumed silica, organic lipophilic gelling agents, such as polyamide-type polycondensates, for instance ethylenediamine/stearyl dimer dilinoleate copolymer, elastomeric organopolysiloxanes, ethylcellulose, silicone-comprising polyamides, galactomannans, block copolymers of “diblock”, “triblock” or “radial” type, of the polystyrene/polyisoprene or polystyrene/polybutadiene type, esters of dextrin and of a fatty acid, such as dextrin palmitates, fatty alcohols, such as behenyl alcohol, cetyl alcohol or stearyl alcohol, hydrogenated plant oils, such as hydrogenated castor oil, glyceryl stearate, and mixtures thereof.

According to one embodiment, a composition of the invention may comprise at least one lipophilic gelling agent selected, in particular, from bentones, polyamide-type polycondensates, for instance ethylenediamine/stearyl dimer dilinoleate copolymer, fatty alcohols, hydrogenated plant oils, and mixtures thereof.

According to one preferred embodiment, the lipophilic gelling agent is selected from bentones, esters of dextrin and of a fatty acid, such as dextrin palmitates, fatty alcohols, in particular behenyl alcohol, glyceryl stearate, and mixtures thereof.

According to one embodiment, a composition of the invention may comprise from 2% to 40% by weight of lipophilic gelling agents, in particular from 3% to 30% by weight, and more particularly from 4% to 20% by weight of lipophilic gelling agents relative to the total weight of the composition.

Semicrystalline Polymers

The term “polymer” is intended to mean compounds comprising at least two repeating units, preferably at least 3 repeating units, and more especially at least 10 repeating units.

The term “semicrystalline polymer” is intended to mean polymers comprising a crystallizable portion, a crystallizable pendent chain or a crystallizable block in the backbone, and an amorphous portion in the backbone, and having a first-order reversible phase-change temperature, in particular of melting (solid-liquid transition). When the crystallizable portion is in the form of a crystallizable block of the polymer backbone, the amorphous portion of the polymer is in the form of an amorphous block; in this case, the semicrystalline polymer is a block copolymer, for example, of the diblock, triblock or multiblock type, comprising at least one crystallizable block and at least one amorphous block.

The term “block” is generally intended to mean at least 5 identical repeating units. The crystallizable block(s) is (are) then of chemical nature different from that of the amorphous block(s).

The semicrystalline polymer has a melting point of greater than or equal to 30° C. (in particular ranging from 30° C. to 80° C.), preferably ranging from 30° C. to 60° C. This melting point is a first-order change of state temperature. This melting point may be measured by any known method, and in particular using a differential scanning calorimeter (DSC).

Advantageously, the semicrystalline polymer(s) to which the invention applies has/have a number-average molecular mass of greater than or equal to 1000. Advantageously, the semicrystalline polymer(s) of the composition of the invention has (have) a number-average molecular mass M̅n ranging from 2000 to 800 000, preferably from 3000 to 500 000, better still from 4000 to 150 000, in particular less than 100 000, and better still from 4000 to 99 000. Preferably, they have a number-average molecular mass of greater than 5600, ranging, for example, from 5700 to 99 000. For the purpose of the invention, the term “crystallizable chain or block” is intended to mean a chain or block which, if it were alone, would reversibly change from the amorphous state to the crystalline state, depending on whether the system is above or below the melting point. For the purpose of the invention, a chain is a group of atoms, which is pendent or lateral relative to the backbone of the polymer. A block is a group of atoms belonging to the backbone, this group constituting one of the repeating units of the polymer. Advantageously, the “crystallizable pendent chain” may be a chain containing at least 6 carbon atoms.

The semicrystalline polymer may be selected from block copolymers comprising at least one crystallizable block and at least one amorphous block, and homopolymers and copolymers bearing at least one crystallizable side chain per repeating unit, and blends thereof. Such polymers are described, for example, in document EP 1 396 259.

According to one more particular embodiment of the invention, the polymer is derived from a monomer containing a crystallizable chain selected from saturated C₁₄ to C₂₂ alkyl (meth)acrylates.

By way of particular example of a structuring semicrystalline polymer that can be used in the composition according to the invention, mention may be made of the products Intelimer® from the company Landec, described in the brochure “Intelimer® polymers”, Landec IP22. These polymers are in solid form at ambient temperature (25° C.), and bear crystallizable side chains.

These semicrystalline polymers can be used in any proportion.

Film Formers

A composition according to the invention may comprise at least one film former.

A film former may be a film-forming polymer.

The term “film-forming” polymer is intended to mean a polymer capable of forming, by itself or in the presence of a film-forming auxiliary, a macroscopically continuous film which adheres to a support, in particular to keratin materials, and preferably a cohesive film, and even better a film of which the cohesion and the mechanical properties are such that said film can be isolated from said support and handled separately, for example when said film is prepared by casting on an anti-adhesive surface such as a Teflon or silicone surface.

Among the film-forming polymers that can be used in the composition of the present invention, mention may be made of synthetic polymers, of free-radical type or polycondensate type, polymers of natural origin, and blends thereof.

As film-forming polymer, mention may in particular be made of acrylic polymers, polyurethanes, polyesters, polyamides, polyureas and cellulosic polymers such as nitrocellulose.

In one embodiment, the film-forming polymer is at least one polymer selected from the group comprising:
film-forming polymers which are soluble in the liquid fatty phase, in particular fat-soluble polymers,

film-forming polymers which are dispersible in the liquid fatty phase, in particular polymers in the form of non-aqueous dispersions of polymer particles, in particular dispersions in silicone oils or hydrocarbon-based oils; in one embodiment, non-aqueous polymer dispersions comprise surface-stabilized polymer particles as mentioned above;
aqueous dispersions of particles of film-forming polymers, often referred to as “latex”; in this case, the composition should include, in addition to the liquid fatty phase, an aqueous phase;
water-soluble film-forming polymers; in this case, the composition should include, in addition to the liquid fatty phase, an aqueous phase.

A film-forming polymer may be dispersed in the form of solid particles in an aqueous phase of the composition, or else solubilized or dispersed in the form of solid particles in a liquid fatty phase. The composition may comprise a blend of these polymers. When the film-forming polymer is in the form of solid particles, these particles may have an average particle size ranging from 5 nm to 600 nm, and preferably from 20 nm to 300 nm.

The term “free-radical film-forming polymer” is intended to mean a polymer obtained by polymerization of unsaturated monomers, in particular ethylenically unsaturated monomers, each monomer being capable of homopolymerization (in contrast to polycondensates).

The film-forming polymers of free-radical type may in particular be vinyl polymers or copolymers, in particular acrylic polymers.

The film-forming vinyl polymers may result from the polymerization of ethylenically unsaturated monomers having at least one acid group and/or esters of these acidic monomers and/or amides of these acidic monomers.

As a monomer which carries an acid group, use may be made of α,β-ethylenic unsaturated carboxylic acids such as acrylic acid, methacrylic acid, crotonic acid, maleic acid or itaconic acid. Use is preferably made of (meth)acrylic acid and crotonic acid, and more preferably (meth)acrylic acid.

The esters of acidic monomers are advantageously selected from esters of (meth)acrylic acid (also called (meth)acrylates), especially alkyl (meth)acrylates, in particular C₁-C₃₀, preferably C₁-C₂₀, alkyl (meth)acrylates, aryl (meth)acrylates, in particular C₆-C₁₀ aryl (meth)acrylates, and hydroxyalkyl (meth)acrylates, in particular C₂-C₆ hydroxyalkyl (meth)acrylates.

Among the alkyl (meth)acrylates, mention may be made of methyl methacrylate, ethyl methacrylate, butyl methacrylate, isobutyl methacrylate, 2-ethylhexyl methacrylate, lauryl methacrylate and cyclohexyl methacrylate.

Among the hydroxyalkyl (meth)acrylates, mention may be made of hydroxyethyl acrylate, 2-hydroxypropyl acrylate, hydroxyethyl methacrylate and 2-hydroxypropyl methacrylate.

Among the aryl (meth)acrylates, mention may be made of benzyl acrylate and phenyl acrylate.

Particularly preferred (meth)acrylic acid esters are alkyl (meth)acrylates.

The alkyl group of the esters may be either fluorinated or perfluorinated, i.e. some or all of the hydrogen atoms of the alkyl group are substituted with fluorine atoms.

As amides of acidic monomers, mention may, for example, be made of (meth)acrylamides, and especially N-alkyl(meth)acrylamides, in particular with C₂-C₁₂ alkyl. Among the N-alkyl(meth)acrylamides, mention may be made of N-ethylacrylamide, N-tert-butylacrylamide, N-tert-octylacrylamide and N-undecylacrylamide.

The film-forming vinyl polymers may also result from the homopolymerization or copolymerization of monomers selected from vinyl esters and styrene monomers. In particular, these monomers may be polymerized with acidic monomers and/or esters thereof and/or amides thereof, such as those mentioned above.

As examples of vinyl esters, mention may be made of vinyl acetate, vinyl neodecanoate, vinyl pivalate, vinyl benzoate and vinyl tert-butylbenzoate.

As styrene monomers, mention may be made of styrene and alpha-methylstyrene.

It is possible to use any monomer known to those skilled in the art that falls within the categories of acrylic and vinyl monomers (including monomers modified with a silicone chain).

Among the film-forming polycondensates, mention may be made of polyurethanes, polyesters, polyesteramides, polyamides, epoxy ester resins and polyureas.

The polyurethanes may be selected from anionic, cationic, nonionic or amphoteric polyurethanes, polyurethane-acrylics, polyurethane-polyvinylpyrrolidones, polyester-polyurethanes, polyureas, polyurea-polyurethanes, and mixtures thereof.

The polyesters may be obtained, in a known manner, by polycondensation of dicarboxylic acids with polyols, in particular diols.

The dicarboxylic acid may be aliphatic, alicyclic or aromatic. As examples of such acids, mention may be made of: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid and 2,6-naphthalenedicarboxylic acid. These dicarboxylic acid monomers may be used alone or as a combination of at least two dicarboxylic acid monomers. Among these monomers, phthalic acid, isophthalic acid and terephthalic acid are preferentially selected.

The diol may be selected from aliphatic, alicyclic and aromatic diols. Use is preferably made of a diol selected from: ethylene glycol, diethylene glycol, triethylene glycol, 1,3-propanediol, cyclohexanedimethanol and 4-butanediol. As other polyols, use may be made of glycerol, pentaerythritol, sorbitol and trimethylol propane.

The polyesteramides can be obtained in a manner analogous to the polyesters, by polycondensation of diacids with diamines or aminoalcohols. Ethylenediamine, hexamethylenediamine, meta-phenylenediamine or para-phenylenediamine may be used as diamine. Monoethanolamine may be used as aminoalcohol.

The polyester may also comprise at least one monomer carrying at least one —SO₃M group, with M representing a hydrogen atom, an ammonium ion NH₄⁺ or a metal ion, for instance a Na⁺, Li⁺, K⁺, Mg²⁺, Ca²⁺, Cu²⁺, Fe²⁺ or Fe³⁺ ion. A bifunctional aromatic monomer comprising such an —SO₃M group may in particular be used.

The aromatic ring of the bifunctional aromatic monomer also carrying an —SO₃M group as described above may be selected, for example, from benzene, naphthalene, anthracene, diphenyl, oxydiphenyl, sulphonyldiphenyl and methylenediphenyl rings. As an example of a bifunctional aromatic monomer also carrying an —SO₃M group, mention may be made of: sulphoisophthalic acid, sulphoterephthalic acid, sulphophthalic acid and 4-sulphonaphthalene-2,7-dicarboxylic acid.

Use is preferably made of copolymers based on isophthalate/sulphoisophthalate, and more particularly copolymers obtained by condensation of diethylene glycol, cyclohexanedimethanol, isophthalic acid or sulphoisophthalic acid.

The optionally modified polymers of natural origin may be selected from shellac resin, sandarac gum, dammars, elemis, copals, cellulosic polymers, and blends thereof.

According to one embodiment of the composition according to the invention, a film-forming polymer may be a water-soluble film-forming polymer and may be present in an aqueous phase of the composition: the composition is therefore solubilized in the aqueous phase of the composition.

According to another embodiment, the film-forming polymer may be present in the form of particles in aqueous dispersion, generally known as latex or pseudolatex. The techniques for preparing these dispersions are well known to those skilled in the art.

As an aqueous film-forming polymer dispersion it is possible to use the acrylic dispersions sold under the names Neocryl XK-90®, Neocryl A-1070®, Neocryl A-1090®, Neocryl BT-62®, Neocryl A-1079®, Neocryl A-523® by the company DSM Neoresins, Dow Latex 432® by the company Dow Chemical, Daitosol 5000 ADO by the company Daito Kasey Kogyo; Syntran 5760, Syntran 5190 and Syntran 5170, sold by the company Interpolymer, or else the aqueous polyurethane dispersions sold under the names Neorez R-981® and Neorez R-974® by the company DSM Neoresins, the products Avalure UR-405®, Avalure UR-410®, Avalure UR-425®, Avalure UR-450® Sancure 875®, Sancure 861®, Sancure 878®, and Sancure 2060® by the company Noveon, Impranil 85® by the company Bayer, and Aquamere H-1511® by the company Hydromer.

As an aqueous film-forming polymer dispersion it is also possible to use the polymer dispersions resulting from the free-radical polymerization of one or more free-radical monomers within and/or partially at the surface, of pre-existing particles of at least one polymer selected from the group consisting of polyurethanes, polyureas, polyesters, polyesteramides and/or alkyds. These polymers are generally referred to as hybrid polymers.

According to another variant embodiment of the composition according to the invention, the film-forming polymer may be present in a liquid fatty phase comprising organic solvents or oils such as those described above.

Preferably, the liquid fatty phase comprises a volatile oil, optionally as a mixture with a non-volatile oil, it being possible for the oils to be selected from the oils mentioned above.

According to another embodiment of the composition according to the invention, the film-forming polymer may be present in the form of surface-stabilized particles that are dispersed in the liquid fatty phase.

The dispersion of surface-stabilized polymer particles may be produced as described in document EP-A-749 747.

The polymer particles may be surface-stabilized by virtue of a stabilizer which may be a block polymer, a graft polymer and/or a random polymer, alone or as a blend.

Dispersions of film-forming polymer in the liquid fatty phase, in the presence of stabilizers, are in particular described in documents EP-A-749 746, EP-A-923 928 and EP-A-930 060.

The size of the polymer particles in dispersion, either in the aqueous phase or in the liquid fatty phase, may range from 5 nm to 600 nm, and preferably from 20 nm to 300 nm.

According to another embodiment of the composition according to the invention, the film-forming polymer may be solubilized in the liquid fatty phase, in which case the film-forming polymer is said to be a fat-soluble polymer.

By way of example of fat-soluble polymers, mention may be made of copolymers of a vinyl ester (the vinyl group being joined directly to the oxygen atom of the ester group, and the vinyl ester having a linear or branched, saturated hydrocarbon-based radical of 1 to 19 carbon atoms attached to the carbonyl of the ester group) and of at least one other monomer which may be a vinyl ester (different from the vinyl ester already present), an α-olefin (having from 8 to 28 carbon atoms), an alkyl vinyl ether (the alkyl group of which contains from 2 to 18 carbon atoms) or an allyl or methallyl ester (having a linear or branched, saturated hydrocarbon-based radical of 1 to 19 carbon atoms attached to the carbonyl of the ester group).

These copolymers may be crosslinked with crosslinkers which may be either of the vinyl type or of the allyl or methallyl type, such as tetraallyloxyethane, divinylbenzene, divinyl octanedioate, divinyl dodecanedioate and divinyl octadecanedioate.

As examples of these copolymers, mention may be made of the following copolymers: vinyl acetate//allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/octadec-1-ene, vinyl acetate/dodec-1-ene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% of divinylbenzene, vinyl dimethylpropionate/vinyl laurate, crosslinked with 0.2% of divinylbenzene, vinyl acetate/octadecyl vinyl ether, crosslinked with 0.2% of tetraallyloxyethane, vinyl acetate/allyl stearate, crosslinked with 0.2% of divinylbenzene, vinyl acetate/octadec-1-ene, crosslinked with 0.2% of divinylbenzene, and allyl propionate/allyl stearate, crosslinked with 0.2% of divinylbenzene.

As fat-soluble film-forming polymers, mention may also be made of fat-soluble homopolymers, and in particular those resulting from the homopolymerization of vinyl esters having from 9 to 22 carbon atoms, or alkyl acrylates or methacrylates, the alkyl radicals having from 10 to 20 carbon atoms.

Such fat-soluble homopolymers may be selected from polyvinyl stearate, polyvinyl stearate crosslinked using divinylbenzene, diallyl ether or diallyl phthalate, polystearyl (meth)acrylate, polyvinyl laurate, polylauryl (meth)acrylate, it being possible for these poly(meth)acrylates to be crosslinked using ethylene glycol dimethacrylate or tetraethylene glycol dimethacrylate.

The fat-soluble homopolymers and copolymers defined above are known and are described in particular in application FR-A-2 262 303; they may have a weight-average molecular weight ranging from 2000 to 500 000, and preferably from 4000 to 200 000.

As fat-soluble film-forming polymers that can be used in the invention, mention may also be made of polyalkylenes, and in particular copolymers of C₂-C₂₀ alkenes, such as polybutene, alkylcelluloses with a saturated or unsaturated, linear or branched C₁ to C₈ alkyl radical, such as ethylcellulose and propylcellulose, ethyl guar and propyl guar, copolymers of vinylpyrrolidone (abbreviated VP below) and in particular copolymers of vinylpyrrolidone and of a C₂ to C₄₀, and better still C₃ to C₂₀, alkene. By way of example of a VP copolymer that can be used in the invention, mention may be made of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene and VP/acrylic acid/lauryl methacrylate copolymer.

As fat-soluble film-forming polymers that can be used in the invention, mention may also be made of silicone resins such as siloxysilicates, which are solid or liquid at ambient temperature.

Among the solid siloxysilicates, mention may be made of the trimethylsiloxysilicates of formula [(CH₃)₃SiO]ₓ(SiO_{4/2})_{y} (MQ units) in which x and y are integers ranging from 50 to 80. As siloxysilicate resins, mention may be made of trimethylsiloxysilicate (TMS) resins optionally in powder form. Such resins are sold under the reference SR1000 by the company General Electric or under the reference TMS 803 by the company Wacker. Mention may also be made of the trimethylsiloxysilicate resins sold in a solvent such as cyclomethicone, sold under the name “KF-7312J” by the company Shin-Etsu, or “DC 749” or “DC 593” by the company Dow Corning.

As liquid silicone resins comprising siloxysilicate MQ units, mention may also be made of arylalkylsiloxysilicate resins, such as phenylpropyldimethylsiloxysilicate (Silshine 151 sold by the company General Electric); the viscosity thereof is between 280 and 600 cSt at 25° C. The preparation of such resins is described in particular in U.S. Pat. No. 5,817,302.

The composition according to the invention may comprise a film-forming auxiliary promoting the formation of a film with the film-forming polymer. Such a film-forming agent may be selected from all of the compounds that are known to those skilled in the art to be capable of fulfilling the desired function, and in particular may be selected from plasticizers and coalescers.

According to one embodiment, a composition of the invention may comprise at least one film former, in particular selected from vinyl polymers or copolymers, polyurethanes, polyesters, polyesteramides, polyamides, epoxy ester resins, polyureas, polymers of natural origin, acrylic dispersions, a water-soluble film-forming polymer, a fat-soluble film-forming polymer, and mixtures thereof.

According to one embodiment, a composition of the invention may comprise at least one film former, in particular selected from vinyl acetate/allyl stearate copolymer, polyvinyl laurate, polyvinyl alcohols, and mixtures thereof.

According to one embodiment, a composition of the invention may comprise from 0.1% to 45% by weight of film former, in particular from 1% to 15% by weight, and more particularly from 1% to 10% by weight of film former relative to the total weight of the composition.

Colorants

A composition according to the invention may also comprise at least one colorant.

A cosmetic composition in accordance with the invention may advantageously incorporate at least one colorant selected from organic or inorganic colorants, in particular such as pigments or nacres conventionally used in cosmetic compositions, which may be fat-soluble or water-soluble, materials with a specific optical effect, and mixtures thereof.

The term “pigments” should be understood to mean white or coloured, inorganic or organic particles which are insoluble in an aqueous solution and are intended for colouring and/or opacifying the resulting film.

The pigments may be present in a proportion of from 0.01% to 15% by weight, especially from 0.01% to 10% by weight, and in particular from 0.02% to 5% by weight, relative to the total weight of the cosmetic composition.

As inorganic pigments that can be used in the invention, mention may be made of titanium dioxide, zirconium oxide or cerium oxide, and also zinc oxide, iron oxide or chromium oxide, ferric blue, manganese violet, ultramarine blue and chromium hydrate.

The pigment may also be a pigment having a structure which may be, for example, of sericite/brown iron oxide/titanium dioxide/silica type. Such a pigment is sold, for example, under the reference Coverleaf NS or JS by the company Chemicals and Catalysts and has a contrast ratio of around 30.

The colorant may also comprise a pigment having a structure which may, for example, be of the type of silica microspheres containing iron oxide. An example of a pigment having this structure is that sold by the company Miyoshi under the reference PC BALL PC-LL-100 P, this pigment being composed of silica microspheres containing yellow iron oxide.

Among the organic pigments that can be used in the invention, mention may be made of carbon black, D & C pigments, lakes based on cochineal carmine, on barium, strontium, calcium or aluminium, or else the diketopyrrolopyrroles (DPPs) described in documents EP-A-542 669, EP-A-787 730, EP-A-787 731 and WO-A-96/08537.

According to one embodiment, a composition of the invention may comprise less than 2%, or even less than 1%, or even less than 0.1% by weight of titanium dioxide relative to the total weight of the composition, and in particular a composition of the invention may be devoid of titanium dioxide.

The term “nacres” should be understood to mean iridescent or non-iridescent coloured particles of any shape, which are in particular produced by certain molluscs in their shell or else are synthesized, and which exhibit a colour effect by optical interference.

The nacres may be selected from pearlescent pigments such as titanium mica coated with an iron oxide, titanium mica coated with bismuth oxychloride, titanium mica coated with chromium oxide, titanium mica coated with an organic dye, and pearlescent pigments based on bismuth oxychloride. This may also involve mica particles at the surface of which are superposed at least two successive layers of metal oxides and/or of organic colorants.

By way of example of nacres, mention may also be made of natural mica coated with titanium dioxide, with iron oxide, with natural pigment or with bismuth oxychloride.

Among the nacres available on the market, mention may be made of the nacres Timica, Flamenco or Duochrome (based on mica) sold by the company Engelhard, the Timiron nacres sold by the company Merck, the Prestige mica-based nacres sold by the company Eckart and the synthetic-mica-based Sunshine nacres sold by the company Sun Chemical.

The nacres may more particularly possess a yellow, pink, red, bronze, orange, brown, gold and/or copper colour or glint.

By way of illustration of nacres which can be employed in the context of the present invention, mention may be made in particular of the golden nacres sold in particular by the company Engelhard under the name Brilliant gold 212G (Timica), Gold 222C (Cloisonne), Sparkle gold (Timica), Gold 4504 (Chromalite) and Monarch gold 233X (Cloisonne); the bronze nacres sold in particular by the company Merck under the name Bronze fine (17384) (Colorona) and Bronze (17353) (Colorona) and by the company Engelhard under the name Super bronze (Cloisonne); the orange nacres sold in particular by the company Engelhard under the name Orange 363C (Cloisonne) and Orange MCR 101 (Cosmica) and by the company Merck under the name Passion orange (Colorona) and Matte orange (17449) (Microna); the brown-hued nacres sold in particular by the company Engelhard under the name Nu-antique copper 340XB (Cloisonne) and Brown CL4509 (Chromalite); the copper-glint nacres sold in particular by the company Engelhard under the name Copper 340A (Timica); the red-glint nacres sold in particular by the company Merck under the name Sienna fine (17386) (Colorona); the yellow-glint nacres sold in particular by the company Engelhard under the name Yellow (4502) (Chromalite); the gold-glint red-hued nacres sold in particular by the company Engelhard under the name Sunstone G012 (Gemtone); the pink nacres sold in particular by the company Engelhard under the name Tan opale G005 (Gemtone); the gold-glint black nacres sold in particular by the company Engelhard under the name Nu antique bronze 240 AB (Timica), the blue nacres sold in particular by the company Merck under the name Matte blue (17433) (Microna), the silver-glint white nacres sold in particular by the company Merck under the name Xirona Silver, and the green-golden pinkish orangish nacres sold in particular by the company Merck under the name Indian summer (Xirona), and mixtures thereof.

The cosmetic composition according to the invention may also comprise water-soluble or fat-soluble dyes. The fat-soluble dyes are, for example, Sudan red, DC Red 17, DC Green 6, β-carotene, soya oil, Sudan brown, DC Yellow 11, DC Violet 2, DC orange 5 and quinoline yellow. The water-soluble dyes are, for example, beetroot juice and methylene blue.

The cosmetic composition according to the invention may also contain at least one material with a specific optical effect.

This effect is different from a simple, conventional hue effect, i.e. a unified and stabilized effect of the kind produced by conventional colorants, such as, for example, monochromatic pigments. For the purpose of the invention, the term “stabilized” signifies absence of an effect of variability of colour with the angle of observation or else in response to a temperature change.

For example, this material may be selected from particles having a metallic glint, goniochromatic colouring agents, diffracting pigments, thermochromic agents, optical brighteners, and also fibres, in particular of interference type. Of course, these various materials may be combined so as to provide the simultaneous manifestation of two effects, or even a new effect in accordance with the invention.

The metallic-glint particles that can be used in the invention are in particular selected from:
particles of at least one metal and/or of at least one metal derivative,
particles comprising a single-substance or multi-substance, organic or inorganic substrate, at least partially coated with at least one metal-glint layer comprising at least one metal and/or at least one metal derivative, and
mixtures of said particles.

Among the metals that may be present in said particles, mention may, for example, be made of Ag, Au, Cu, Al, Ni, Sn, Mg, Cr, Mo, Ti, Zr, Pt, Va, Rb, W, Zn, Ge, Te, Se and mixtures or alloys thereof. Ag, Au, Cu, Al, Zn, Ni, Mo, Cr and mixtures or alloys thereof (for example, bronzes and brasses) are preferred metals.

The term “metal derivatives” denotes compounds derived from metals, in particular oxides, fluorides, chlorides and sulphides.

By way of illustration of these particles, mention may be made of aluminium particles, such as those sold under the names Starbrite 1200 EAC® by the company Siberline and Metalure® by the company Eckart.

Mention may also be made of metal powders of copper or of alloy mixtures, such as the references 2844 sold by the company Radium Bronze, metal pigments, such as aluminium or bronze, for instance those sold under the name Rotosafe 700 from the company Eckart, the silica-coated aluminium particles sold under the name Visionaire Bright Silver from the company Eckart, and the metal alloy particles such as silica-coated bronze (copper and zinc alloy) powders sold under the name Visionaire Bright Natural Gold from the company Eckart.

The particles in question may also be particles comprising a glass substrate, such as those sold by the company Nippon Sheet Glass under the name Microglass Metashine.

The goniochromatic colouring agent may be selected, for example, from multilayer interference structures and liquid-crystal colouring agents.

Examples of symmetrical multilayer interference structures that may be used in compositions prepared in accordance with the invention are, for example, the following structures: Al/SiO₂/Al/SiO₂/Al, pigments having this structure being sold by the company Dupont de Nemours; Cr/MgF₂/Al/MgF₂/Cr, pigments having this structure being sold under the name Chromaflair by the company Flex; MoS₂/SiO₂/Al/SiO₂/MoS₂; Fe₂O₃/SiO₂/Al/SiO₂/Fe₂O₃, and Fe₂O₃/SiO₂/Fe₂O₃/SiO₂/Fe₂O₃, pigments having these structures being sold under the name Sicopearl by the company BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona® by the company Merck (Darmstadt). By way of example, these pigments may be the pigments of silica/titanium oxide/tin oxide structure sold under the name Xirona Magic by the company Merck, the pigments of silica/brown iron oxide structure sold under the name Xirona Indian Summer by the company Merck and the pigments of silica/titanium oxide/mica/tin oxide structure sold under the name Xirona Caribbean Blue by the company Merck. Mention may also be made of the Infinite Colors pigments from the company Shiseido. Depending on the thickness and the nature of the various layers, different effects are obtained. Thus, with the Fe₂O₃/SiO₂/Al/SiO₂/Fe₂O₃ structure, the colour changes from green-golden to red-grey for SiO₂ layers of 320 to 350 nm; from red to golden for SiO₂ layers of 380 to 400 nm; from violet to green for SiO₂ layers of 410 to 420 nm; from copper to red for SiO₂ layers of 430 to 440 nm.

By way of example of pigments with a polymeric multilayer structure, mention may be made of those sold by the company 3M under the name Color Glitter.

Examples of liquid-crystal goniochromatic particles that may be used include those sold by the company Chemx, and also the product sold under the name Helicone® HC by the company WACKER.

Fillers

A composition in accordance with the invention may also comprise at least one filler, of organic or inorganic nature, which makes it possible in particular to confer thereon an improved stability with regard to exudation.

The term “filler” should be understood to mean colourless or white solid particles of any shape, which are in a form that is insoluble and dispersed in the medium of the composition. Inorganic or organic in nature, they make it possible to confer body or rigidity on the composition, and/or softness, a matt aspect and uniformity on the makeup.

The fillers used in the compositions according to the present invention may be of lamellar, globular or spherical form, in the form of fibres or in any other form intermediate between these defined forms.

The fillers according to the invention may or may not be surface-coated, and in particular they may be surface-treated with silicones, amino acids, fluoro derivatives, or any other substance that promotes the dispersion and compatibility of the filler in the composition.

For the purpose of the present invention, the terms “mineral fillers” and “inorganic fillers” are used interchangeably.

Among the mineral fillers that can be used in the compositions according to the invention, mention may be made of talc, mica, silica, trimethyl siloxysilicate, kaolin, bentone, precipitated calcium carbonate, magnesium carbonate, magnesium hydrogen carbonate, hydroxyapatite, boron nitride, hollow silicon microspheres (Silica Beads from Maprecos), glass or ceramic microcapsules, silica-based fillers such as Aerosil 200, Aerosil 300; Sunsphere L-31, Sunsphere H-31 sold by Asahi Glass; Chemicelen sold by Asahi Chemical; composites of silica and of titanium dioxide, such as the TSG series sold by Nippon Sheet Glass, and mixtures thereof.

Among the organic fillers that can be used in the compositions according to the invention, mention may be made of polyamide powders (Nylon® Orgasol from Atochem), poly-β-alanine powders and polyethylene powders, polytetrafluoroethylene powders (Teflon®), lauroyllysin, starch, tetrafluoroethylene polymer powders, hollow polymer microspheres such as Expancel (Nobel Industrie), metal soaps derived from organic carboxylic acids containing from 8 to 22 carbon atoms, preferably from 12 to 18 carbon atoms, for example zinc stearate, magnesium stearate or lithium stearate, zinc laurate, magnesium myristate, Polypore® L 200 (Chemdal Corporation), silicone resin microbeads (for example, Tospearl® from Toshiba), polyurethane powders, in particular powders of crosslinked polyurethane comprising a copolymer, said copolymer comprising trimethylol hexyllactone. In particular, it may be a polymer of hexamethylene diisocyanate/trimethylol hexyllactone. Such particles are in particular commercially available, for example, under the name Plastic Powder D-400® or Plastic Powder D-800® from the company TOSHIKI, and mixtures thereof.

Active Agents

A composition of the invention may also comprise at least one cosmetic active agent and/or one dermatological active agent.

By way of non-limiting examples of cosmetic and/or dermatological active agents suitable for the invention, mention may be made of the active agents selected from the following agents:
moisturizers, desquamating agents, barrier function enhancers, depigmenting agents, antioxidants, dermodecontracting agents, anti-glycation agents, agents for stimulating the synthesis of dermal and/or epidermal macromolecules and/or preventing their degradation, agents for stimulating fibroblast or keratinocyte proliferation and/or keratinocyte differentiation, agents for promoting maturation of the horny envelope, NO-synthase inhibitors, peripheral benzodiazepine receptor (PBR) antagonists, agents for increasing sebaceous gland activity, agents for stimulating the energy metabolism of cells, tensioning agents, fat-restructuring agents, slimming agents, agents for promoting cutaneous microcirculation, calmatives and/or anti-irritants, sebum-regulating or anti-seborrhoea agents, astringents, cicatrizing agents, anti-inflammatory agents and anti-acne agents,
matting agents, soft-focus effect fillers, fluorescent agents, agents for promoting the natural pinkish colouring of the skin, and abrasives or exfoliant fillers,
and mixtures thereof.

The cosmetic and/or dermatological active agents suitable for the invention may in particular be selected from the agents mentioned in application EP 1 847 247.

It is part of the abilities of those skilled in the art to select said active agent(s) and the content thereof as a function of the desired effect on the keratin materials, and so as not to cause any adverse effect on the cosmetic properties of the compositions of the invention.

According to one particular embodiment of the invention, the composition according to the invention comprises at least 70% by weight, preferably at least 80% by weight of ingredients of natural origin, relative to the total weight of said composition.

The term “natural compound” is intended to mean in particular a compound which is obtained directly from the earth or from the soil, or from plants or from animals, via, as appropriate, one or more physical processes, such as, for example, milling, refining, distillation, purification or filtration.

The term compounds “of natural origin” is intended to mean in particular a natural compound having undergone one or more secondary chemical or industrial treatments generating modifications that do not affect the essential qualities of this compound, and/or a compound comprising predominantly natural constituents which may or may not have undergone transformations, as indicated above.

By way of non-limiting example of a secondary chemical or industrial treatment generating modifications that do not affect the essential qualities of a natural compound, mention may be made of those authorized by control organizations such as Ecocert (system of reference for biological and ecological cosmetic products, January 2003) or defined in the manuals recognized in the field, such as “Cosmetics and Toiletries Magazine”, 2005, vol. 120, 9:10.

Additives

A cosmetic composition according to the invention may also further comprise any additive normally used in the field under consideration, for example selected from antioxidants, fibres, essential oils, preservatives, fragrances, neutralizing agents, anti-UV protective agents, vitamins, moisturizers, cosmetic active agents, and mixtures thereof.

Those skilled in the art can adjust the nature and amount of the additives present in the compositions in accordance with the invention by means of routine operations, such that the cosmetic properties and the stability properties desired for these compositions are not thereby affected.

A composition according to the invention may in particular be in the form of a composition for making up and/or caring for keratin fibres, especially the eyelashes, in particular a mascara.

More particularly, a composition of the invention may be a cream mascara.

A composition of the invention may be obtained by means of any method of preparation known to those skilled in the art.

Applicator

An applicator included in the makeup and/or care assembly according to the present invention comprises means for smoothing and/or separating the eyelashes or the eyebrows.

It comprises in particular a plurality of application members arranged on a support in the form of at least one row whose length is such that the application members can at best simultaneously contact at least a part of the eyelashes or of the eyebrows of the fringe, in order to smooth them out and/or to separate them.

According to a preferred embodiment, the applicator according to the invention comprises:
a shaft,
a support connected to the shaft and extending along a longitudinal axis, and
a plurality of application members, being only two in number, where appropriate, arranged on the support and extending substantially transversely to the longitudinal axis of the support.

Thus, according to one most particularly preferred embodiment, the applicator comprises a plurality of application members arranged on a support in the form of at least one row whose length is such that the application members can simultaneously contact a part of the eyelashes or of the eyebrows of the fringe.

The application members can extend over a distance along the longitudinal axis of the support, which is such that the application members can at best simultaneously contact more than a quarter of the eyelashes or of the eyebrows of the fringe, while the longitudinal axis of the support is oriented substantially tangentially to the fringe.

A portion of made-up eyelashes or eyebrows may, for example, correspond to more than a quarter of the length of the fringe.

The application members may comprise teeth and/or bristles.

This applicator makes it possible to conveniently deposit the composition on the eyelashes or the eyebrows while at the same time exerting a possible combing action.

This applicator makes it possible to deposit, in a sustained manner, material on the eyelash or eyebrow fringe to be made up, preferably the portion which is at the exterior of the eyelash or eyebrow fringe.

The application members may, for example, extend on the support over a distance of between approximately 0.1 and 3 cm, for example less than or equal to 2.5 cm.

The applicator included in the assembly according to the present invention may comprise:
a shaft,
a support connected to the shaft and extending along a longitudinal axis,
at least one row of application members, in particular constituted of teeth or bristles, arranged on the support, the application members of the row each extending substantially transversely to the longitudinal axis of the support and being able to have free ends whose distance to the longitudinal axis varies non-monotonously on moving between the extreme application members of the row.

For the purpose of the present invention, the expression “varies non-monotonously” should be understood to mean that the line connecting the free ends of the application members has a distance to the longitudinal axis of the support that passes through at least one extremum, for example a maximum.

The presence of a maximum in the row of application members may allow gradual penetration of the application members into the eyelashes or the eyebrows and thus facilitate the making up of a portion of eyelash or eyebrow fringe within the meaning of the invention.

The number of application members of the row, in particular the number of teeth, may, for example, be between 3 and 9, preferably between 3 and 8, in particular between 3 and 7.

According to one embodiment, the support and the application members are made as a single piece, in particular by moulding or machining As a variant, the application members may be connected to the support.

The support and the shaft may be made as a single piece, in particular by moulding, or, as a variant, the support may be connected to the shaft. In this case, the support may comprise a tip arranged so as to engage in a housing of the shaft, or the shaft may comprise a tip arranged so as to engage in a housing of the support.

The shaft may extend along a longitudinal axis different from the longitudinal axis of the support.

In this case, the longitudinal axis of the shaft may form, with the longitudinal axis of the support, in particular for the portion thereof that bears the application members, an angle of between 5° and 45°, in particular between 15° and 25°, for example equal to 20°, approximately. This configuration may allow more comfortable handling of the applicator during the application of a makeup composition in accordance with the invention to the eyelashes or the eyebrows. The longitudinal axis of the support may, for example, be curvilinear or rectilinear.

As a variant, the shaft may extend along a longitudinal axis that is identical to the longitudinal axis of the support.

The applicator and/or the support may be at least partially made of a supple or rigid material, in particular at least partially of thermoplastic, elastomeric or elastomeric-thermoplastic material, in particular by moulding. It would not constitute a departure from the context of the present invention if the applicator were at least partially made of wood or of metal or of yet other materials, in particular by machining.

When the support is made of an elastically deformable material, this may make it possible to perform preferential wiping of one side of the support.

The shaft and the support may or may not be made of different materials.

When the applicator is observed from the side, a line connecting the free ends of the application members may have a distance to the longitudinal axis of the support that passes through at least one extremum, in particular a maximum, which may make the penetration of the applicator into the eyelashes more gradual.

The abovementioned line may in particular have, at least partially, a pointed, in particular triangular, shape or a rounded, in particular circular or ogival, shape.

Whatever the profile of the abovementioned line, when there is a maximum and a minimum, the angle formed between, on the one hand, the straight line connecting the free ends of an application member of greater length associated with this maximum and of an application member of smaller length associated with the closest minimum of the application member of longer length, and, on the other hand, the normal to the longitudinal axis of the support, this normal passing through the free end of the application member of greater length, is for example between 25° and 60° approximately, in particular between 25° and 50°, in particular between 25° and 45°, for example between 30° and 45°, for example equal to approximately 43°.

The distance from the abovementioned line to the longitudinal axis of the support may also pass through two, or even more, extremes.

The application members may be arranged in a single row or in at least two rows. In the latter case, the application members of a first row and those of a second row may extend in different directions or, as a variant, in parallel directions. The two rows may be, for example, arranged substantially opposite each other relative to the support. The height of the application members may, for example, be between 0.5 and 10 mm approximately, and in particular between 1 and 3 mm approximately.

When the application members are arranged in at least one row, this row may comprise a succession of application members extending alternately, at least partially, on either side of a geometrical separating surface.

The application members may or may not have aligned bases.

When the application members are arranged in at least one row, the row may extend along a non-rectilinear axis. The application members may also be made alternately on either side of a median core.

Again when the application members are arranged in at least one row, this row may comprise a succession of application members located alternately, at least partially, on either side of a geometrical separating surface, two consecutive application members having adjacent parts that are connected or that overlap when the applicator is observed from the side, and forming between them a groove for receiving therein an eyelash or an eyebrow.

The applicator may comprise a comb or a brush.

When the applicator comprises a brush, the support may comprise two twisted wires of small diameter, in particular less than 0.7 mm, for example between 0.2 and 0.6 mm, better still between 0.35 and 0.5 mm.

Still in the case where the applicator comprises a brush, the application members may be bristles of small diameter, for example less than or equal to 8/100 mm, better still 6/100 mm.

When the brush comprises a twisted core, the number of turns of the brush may be between 3 and 6, for example. In the case of a brush with a twisted core, the bristles extend from the core in two helical courses. The number of turns of the brush corresponds to the sum of the revolutions around the core of the two courses. A part of the twisted core may be devoid of bristles, in particular close to the junction between the support and the shaft. The cross section of the envelope surface of the brush may be circular, square or another shape.

The brush may have a biconical envelope surface. The envelope surface may also be conical or frustoconical with the base of the cone located on the side of the distal or proximal end of the brush.

The applicator included in the assembly according to the invention may take the form of a brush for the application of a product of the eyelashes or the eyebrows, comprising a twisted core and bristles extending from the core.

The applicator included in the assembly according to the invention may comprise a core and bristles extending from the core, contained in an envelope surface of substantially frustoconical, conical or biconical shape, in which the smallest angle formed between a plane containing the largest cross section of the brush and a tangent to the envelope surface of the brush at the intersection with this plane is between 20° and 60°, in particular between 25° and 50°, for example between 30° and 35°.

The applicator included in the assembly according to the invention may comprise a core and bristles extending from the core, in which the envelope surface of the brush has a substantially biconical shape, with an apex angle at the junction of the cone frustums of less than or equal to 120°, or even less than or equal to 90°.

The applicator included in the assembly according to the invention may comprise a support and teeth extending transversally to the longitudinal axis of the support, comprising a tooth of greater length having a first length and a tooth of shorter length having a second length, in which comb the ratio of the first length to the second length is greater than or equal to 1.3.

The applicator included in the assembly according to the invention may comprise a support and teeth extending transversally to the longitudinal axis of the support, in which the angle formed between a straight line joining the free end of a tooth of shorter length and the free end of a tooth of greater length, and the perpendicular to the longitudinal axis of the support passing through the free end of the tooth of greater length, is between 20° and 60°.

The applicator according to the invention may be in the form of a brush comprising an arrangement of bristles held by a twisted wire. Such a twisted brush is described in particular in U.S. Pat. No. 4,887,622. It may also be in the form of a comb comprising a plurality of application members, obtained in particular by moulding. Such combs are described, for example, in patent FR 2 796 529.

The composition according to the invention may be packaged in a container delimiting at least one compartment which comprises said composition, said container being closed by means of a closing member.

The container may be associated with the applicator.

The applicator may be solidly attached to the container, as described, for example, in patent FR 2 761 959. Advantageously, the applicator is solidly attached to a shaft which is itself solidly attached to the closing member.

The closing member may be coupled to the container by screwing. Alternatively, the coupling between the closing member and the container occurs other than by screwing, in particular via a bayonet mechanism, by click-fastening or by tightening. The term “click-fastening” is intended to mean in particular any system involving the passing of a rim or bead of material by elastic deformation of a portion, in particular of the closing member, followed by return to the elastically unstressed position of said portion after the rim or bead has been passed.

The container may be at least partly made of thermoplastic material. By way of examples of thermoplastic materials, mention may be made of polypropylene or polyethylene.

Alternatively, the container is made of a non-thermoplastic material, in particular of glass or of metal or alloy.

The container is preferably equipped with a drainer located in the region of the aperture of the container. Such a drainer makes it possible to wipe the applicator and, optionally, the shaft to which it may be solidly attached. Such a drainer is described, for example, in patent FR 2 792 618.

The applicator or the container according to the invention may have vibrating and/or rotating and/or heating means.

The present invention will be understood more clearly by means of the examples which follow.

These examples are presented merely by way of illustration of the invention and should not be interpreted as limiting the scope thereof.

### EXAMPLES

### Example 1

### Waterproof Mascara

FORMULA IA

| Trade name | Chemical name | Amount (%) |
|---|---|---|
| HC 1720 | Rice bran wax | 2.2 |
| Candellilla Wax SP 75 | Carnauba wax | 8.4 |
| Cerabeil BIO-D | Certified biological beeswax | 8.2 |
| Mexomere PQ | Vinyl acetate/allyl stearate | 6.7 |
| | copolymer | |
| Mexomere PP | Polyvinyl laurate | 0.7 |
| Uniclear 100 VG | Ethylenediamine/stearyl | 1 |
| | dimer dilinoleate copolymer | |
| Sunpuro black iron oxide | Black iron oxide | 5.14 |
| REMY DR I | Irradiation-decontaminated | 1 |
| | rice starch | |
| Cetiol UT | Undecane/tridecane | 53.6 |
| Bentone 38 VCG | Disteardimonium hectorite | 5 |
| Arconate propylene | Propylene carbonate | 1.6 |
| carbonate | | |
| Distilled water | Distilled water | 6.14 |
| Celvol 540 PV alcohol | Polyvinyl alcohol | 0.32 |
| | | 100 |

Preparation

The waxes, the Uniclear, the iron oxides and the rice starch are mixed with heating at 95° C. in order to obtain the fatty phase.

The Cetiol UT, the hectorite and the propylene carbonate are mixed at ambient temperature in order to obtain the solvent phase.

The water and the polyvinyl alcohol are mixed at ambient temperature in order to obtain the aqueous phase.

The fatty phase is then mixed with the solvent phase with vigorous stirring.

Finally, the aqueous phase and the Mexomere PP and PQ are added at ambient temperature.

Aesthetic Flash Test (7 Models)

A comparative example is prepared.

Formula IB

| Ingredient | Formula IB |
|---|---|
| Modified distearyl dimethylammonium hectorite | 5.8 |
| (Bentone 38 VCG) | |
| Talc | 1 |
| Black iron oxide | 4.2 |
| Preservatives | Qs |
| Carnauba wax (Candellilla Wax SP 75) | 4.7 |
| Beeswax | 8.3 |
| Protected refined paraffin | 2.8 |
| Hydrogenated jojoba oil | 0.1 |
| Rice bran wax | 2.8 |
| Microdispersion of carnauba wax with 10% ethanol | 7 |
| (MEXORYL SAP) | |
| Polyvinyl laurate (MEXOMERE PP) | 2.2 |
| Vinyl acetate/ally stearate copolymer (65/35) | 3.3 |
| (MEXOMERE PQ) | |
| Vinylpyrrolidone/eicosene copolymer (ANTARON V220) | 2 |
| Ethylenediamine/stearyl dimer dilinoleate copolymer | 1 |
| (UNICLEAR 100 VG) | |
| Ethanol | 2 |
| Propylene carbonate | 1.9 |
| Isododecane | Qs 100 |

The composition according to formula IA was compared with formula IB with a lengthening gesture on 6 models and a volume gesture on 1 model.

With the lengthening gesture (30 brushstrokes), the 2 formulae are similar on application: the makeup result is a lengthening, curling effect, the eyelashes are smooth with an even fringe.

On the other hand, with the volume gesture (60 brushstrokes), the formula IB reveals less attachment as it is applied, which is reflected by a mediocre volumizing effort, whereas formula IA (invention) results in a strong volumizing effect.

The mascara according to formula IA also makes it possible to obtain a makeup result with an intense colour, and makes the eyelashes more supple to the touch.

Moreover, its long drying time allows comfortable application. It also confers good staying power through the day.

### Example 2

Waterproof Mascara

| Trade name | Chemical name | Amount (%) |
|---|---|---|
| HC 1720 | Rice bran wax | 2.2 |
| Candellilla Wax SP 75 | Carnauba wax | 8.4 |
| Cerabeil BIO-D | Certified biological beeswax | 8.2 |
| Mexomere PQ | Vinyl acetate/allyl stearate | 6.7 |
| | copolymer | |
| Mexomere PP | Polyvinyl laurate | 0.7 |
| Uniclear 100 VG | Ethylenediamine/stearyl | 1 |
| | dimer dilinoleate copolymer | |
| Sunpuro black iron oxide | Black iron oxide | 5.14 |
| REMY DR I | Irradiation-decontaminated | 1 |
| | rice starch | |
| Cetiol UT | Undecane/tridecane | Qs 100 |
| Cutina GMS V | Glyceryl stearate | 11 |
| | Alcohol | 3 |
| Distilled water | Distilled water | 6.14 |
| Celvol 540 PV alcohol | Polyvinyl alcohol | 0.32 |

The mascara is prepared as indicated above.

### Example 3

Waterproof Mascara

| | | IIIA | IIIB |
|---|---|---|---|
| | | invention | comparative |
| Trade name | Chemical name | Amount (%) | Amount (%) |
| Candellilla Wax SP 75 | Carnauba wax | 4.73 | 4.73 |
| Carnauba wax (and) | Carnauba wax | 7 | 7 |
| PEG-30 glyceryl | (and) PEG-30 | | |
| stearate | glyceryl stearate | | |
| Beeswax | Beeswax | 4.89 | 4.89 |
| Synthetic beeswax | Synthetic beeswax | 3.38 | 3.38 |
| Mexomere PQ | Vinyl acetate/allyl | 2.21 | 2.21 |
| | stearate copolymer | | |
| Mexomere PP | Polyvinyl laurate | 0.75 | 0.75 |
| VP/eicosene | VP/eicosene | 2 | 2 |
| copolymer | copolymer | | |
| Polybutene | Polybutene | 1 | 1 |
| Iron oxides | Iron oxides | 4.19 | 4.19 |
| | Oryza sativa (rice) | 1.5 | 1.5 |
| | starch | | |
| Cetiol UT | Undecane/tridecane | 48.83 | |
| Bentone 38 VCG | Disteardimonium | 5.8 | 5.8 |
| | hectorite | | |
| Arconate propylene | Propylene carbonate | 1.94 | 1.94 |
| carbonate | | | |
| Distilled water | Distilled water | 2.89 | 2.89 |
| Denaturated alcohol | Denaturated alcohol | 2 | 2 |
| Synthetic wax | Synthetic wax | 0.1 | 0.1 |
| Polyethylene | Polyethylene | 2 | 2 |
| Polyquaternium-10 | Polyquaternium-10 | 0.1 | 0.1 |
| | Sodium | 1 | 1 |
| | polymethacrylate | | |
| PEG/PPG 17/18 | PEG/PPG 17/18 | 0.1 | 0.1 |
| dimethicone | dimethicone | | |
| BIS-PEG-12 | BIS-PEG-12 | 1 | 1 |
| dimethicone | dimethicone | | |
| candellilate | candellilate | | |
| Paraffin | Paraffin | 2.28 | 2.28 |
| Panthenol | Panthenol | 0.2 | 0.2 |
| Propylparaben | Propylparaben | 0.1 | 0.1 |
| Methylparaben | Methylparaben | 0.01 | 0.01 |
| Isododecane | Isododecane | | 48.83 |
| | | 100 | 100 |

The mascara is prepared as indicated above.

Aesthetic Flash

The composition according to formula IIIA (invention) was compared with formula IIIB (comparative) with a lengthening gesture on 6 models and a volume gesture on 1 model.

With the lengthening gesture (30 brushstrokes), the 2 formulae are similar on application. As regards the makeup result, the eyelashes are smoother with a tendency to be more supple for the composition IIIA.

With the volume gesture (60 brushstrokes), the formula IIIA slides better on the eyelashes; the eyelashes are even smoother and are also better separated.

With an equivalent waterproof effectiveness for the 2 compositions, the composition IIIA according to the invention also confers an improved makeup result on the eyelashes, with more even charging of the eyelashes on deposition, and smoother eyelashes.

### Example 4

### Waterproof Mascara

| | | IVA | IVB |
|---|---|---|---|
| | | invention | comparative |
| Trade name | Chemical name | Amount (%) | Amount (%) |
| | Oryza sativa (rice) | 2.8 | 2.8 |
| | starch | | |
| Candellilla Wax SP 75 | Carnauba wax | 4.7 | 4.7 |
| | Carnauba wax (and) | 7 | 7 |
| | PEG-30 glyceryl | | |
| | stearate | | |
| Beeswax | Beeswax | 8.3 | 8.3 |
| Mexomere PQ | Vinyl acetate/allyl | 3.3 | 3.3 |
| | stearate copolymer | | |
| Mexomere PP | Polyvinyl laurate | 2.2 | 2.2 |
| Uniclear 100 VG | Ethylenediamine/ | 1 | 1 |
| | stearyl | | |
| | dimer dilinoleate | | |
| | copolymer | | |
| VP/eicosene | VP/eicosene | 2 | 2 |
| copolymer | copolymer | | |
| Iron oxides | Iron oxides | 4.2 | 4.2 |
| Cetiol UT | Undecane/tridecane | 50.56 | |
| Bentone 38 VCG | Disteardimonium | 5.8 | 5.8 |
| | hectorite | | |
| Arconate propylene | Propylene carbonate | 1.9 | 1.9 |
| carbonate | | | |
| Talc | Talc | 1 | 1 |
| Denaturated alcohol | Denaturated alcohol | 2 | 2 |
| Paraffin | Paraffin | 2.8 | 2.8 |
| Hydrogenated jojoba | Hydrogenated | 0.1 | 0.1 |
| oil | jojoba oil | | |
| Propylparaben | Propylparaben | 0.19 | 0.19 |
| Methylparaben | Methylparaben | 0.15 | 0.15 |
| Isododecane | Isododecane | | 50.56 |
| | | 100 | 100 |

The mascara is prepared as indicated above.

Aesthetic Flash

The composition according to formula IVA (invention) was compared with formula IVB (comparative) with a lengthening gesture on 6 models and a volume gesture on 1 model.

With the lengthening gesture (30 brushstrokes), the 2 formulae are similar on application. As regards the makeup result, the eyelashes are smoother with a more intense black colour and a tendency to be more supple for the composition IVA.

With the volume gesture (60 brushstrokes), the formula IVA slides better on the eyelashes; the eyelashes are even smoother and blacker.

With an equivalent waterproof effectiveness for the 2 compositions, the composition IVA according to the invention also confers an improved makeup result for the eyelashes, with a more even charging of the eyelashes on deposition, greater thickness of the deposit (charging effect), smoother eyelashes and greater intensity of the colour.

1. An anhydrous cosmetic composition for making up and/or caring for the eyelashes and/or the eyebrows comprising at least one volatile hydrocarbon solvent of C₉-C₁₅ volatile linear alkane(s) type having an evaporation rate of less than or equal to 0.13 mg/cm²/min, said composition comprising less than 25% by weight of non-volatile oil relative to the total weight of the composition and less than 35% by weight of surfactant relative to the total weight of the composition.

2. Composition according to claim 1, in which said volatile hydrocarbon solvent is a volatile linear alkane or a mixture of volatile linear alkanes, in particular selected from volatile linear alkanes containing from 11 to 13 carbon atoms.

3. Composition according to claim 1, in which said volatile hydrocarbon solvent is a volatile linear alkane of plant origin.

4. Composition according to claim 1, in which said volatile hydrocarbon solvent is a volatile linear alkane selected from n-nonane, n-undecane, n-dodecane, n-tridecane, and mixtures thereof.

5. Composition according to claim 1, comprising from 0.5% to 90% by weight of volatile hydrocarbon solvent of volatile linear alkane(s) type, in particular from 1% to 80% by weight, and more particularly from 5% to 60% by weight of volatile hydrocarbon solvent of volatile linear alkane(s) type relative to the total weight of the composition.

6. Composition according to claim 1, further comprising at least one agent for structuring a liquid fatty phase, selected from waxes, semicrystalline polymers, lipophilic gelling agents, pasty compounds, and mixtures thereof.

7. Composition according to claim 6, in which said lipophilic gelling agent is selected, in particular, from inorganic lipophilic gelling agents such as modified clays, for instance bentones, or fumed silica, organic lipophilic gelling agents, such as polyamide-type polycondensates, for instance ethylenediamine/stearyl dimer dilinoleate copolymer, elastomeric organopolysiloxanes, ethylcellulose, silicone-comprising polyamides, galactomannans, block copolymers of “diblock”, “triblock” or “radial” type, of the polystyrene/polyisoprene or polystyrene/polybutadiene type, esters of dextrin and of a fatty acid, fatty alcohols, such as behenyl alcohol, cetyl alcohol or stearyl alcohol, hydrogenated plant oils, such as hydrogenated castor oil, glyceryl stearate, and mixtures thereof.

8. Composition according to claim 7, comprising from 2% to 40% by weight of lipophilic gelling agents, in particular from 3% to 30% by weight, and more particularly from 4% to 20% by weight of lipophilic gelling agents relative to the total weight of the composition.

9. Composition according to claim 7, said composition further comprising at least one wax selected from candelilla wax, carnauba wax, rice bran wax, beeswax, isomerized jojoba oil, paraffin wax, microcrystalline wax and mixtures thereof.

10. Composition according to claim 9, comprising from 1% to 40% by weight of wax, in particular from 8% to 30%, and more particularly from 15% to 20% by weight of wax relative to the total weight of the composition.

11. Composition according to claim 1, said composition further comprising at least one film former and, optionally, a film-forming auxiliary.

12. Composition according to claim 11, in which said film-forming agent is selected from vinyl acetate/allyl stearate copolymer, polyvinyl laurate, the polyvinyl alcohols, and mixtures thereof.

13. Composition according to claim 11, comprising from 0.1% to 45% by weight of film former, in particular from 1% to 15% by weight, and more particularly from 1% to 10% by weight of film former relative to the total weight of the composition.

14. Composition according to claim 1 further comprising at least one colorant.

15. Composition according to claim 1, said composition being a mascara.

16. Cosmetic process for making up and/or caring for eyelashes, comprising at least the application, to said eyelashes, of at least one layer of a composition as defined in claim 1.

17. Assembly for making up and/or caring for the eyelashes, comprising:
at least one composition as defined in claim 1,
and at least one applicator of the composition, said applicator comprising means for smoothing out and/or separating the eyelashes.
